(19) United States
(12) Patent Application Publication
(10) Pub. No.: US 2017/0351805 A1
(43) Pub. Date: Dec. 7, 2017

(54) METHOD FOR DETERMINING GENOTYPE OF PARTICULAR GENE LOCUS GROUP OR INDIVIDUAL GENE LOCUS, DETERMINATION COMPUTER SYSTEM AND DETERMINATION PROGRAM

(71) Applicant: NATIONAL UNIVERSITY CORPORATION TOHOKU UNIVERSITY, Sendai-shi, Miyagi (JP)

(72) Inventors: Masao NAGASAKI, Sendai-shi (JP); Naoki NARIAI, Sendai-shi (JP); Kaname KOJIMA, Sendai-shi (JP)

(73) Assignee: NATIONAL UNIVERSITY CORPORATION TOHOKU UNIVERSITY, Sendai-shi, Miyagi (JP)

(21) Appl. No.: 15/539,731

(22) PCT Filed: Dec. 25, 2015
(86) PCT No.: PCT/JP2015/086194

§ 371 Date: Jun. 26, 2017

(30) Foreign Application Priority Data
Dec. 26, 2014 (JP) 2014-265704

(51) Int. Cl. G06F 19/18; C12N 15/09; G06F 19/24; C12Q 1/68; C12P 19/34

(52) U.S. Cl. CPC G06F 19/18; G06F 19/24; C12Q 1/6869; C12N 15/09; C12P 19/34; C12Q 1/6881

(57) ABSTRACT

It is an invention aimed at providing methods for optimizing read information mapped to the particular gene loci such as MHC loci under a framework of probabilistic statistical processing. In the present invention, a step in which for all reads, calculation of the expected number of mappings to alleles of the particular gene loci is performed for read information in which mapping of reads to alleles of the particular gene loci is identified, a step in which the total number of expected mappings for each allele is calculated, and a step in which the fraction of reads allocated to each allele is calculated, are repeatedly executed, in which the optimization of read information is performed in a computer, and based on the optimized information, a method of easily and accurately estimating the genotypes of the particular gene loci, a computer system, and a computer program capable of easily and accurately estimating the genotype of the particular gene loci, are provided.

### TECHNICAL FIELD

The present invention relates to the field of nucleic acid-based genetic analysis, and more particularly provides a method of genotyping selected gene loci or an individual gene locus easily and highly sensitively based on read information of human DNA generated from high-throughput sequencers. Preferred examples of the gene loci or individual gene locus include gene loci or an individual gene locus of the major histocompatibility complex (MHC). Human MHC is a human leucocyte antigen (HLA).

### BACKGROUND ART

The genotype (allele) of particular gene loci is associated with various diseases and phenotypes of human and other organisms.

For example, alleles of HLA which is s human MHC are important not only for judgment of compatibility in organ transplantation but also for associations with drug susceptibility, and numerous diseases, such as diabetes, autoimmune diseases, and narcolepsy, which have been recently reported. Therefore, it is extremely important to properly determine the genotype of each HLA locus in HLA loci in clinical applications.

Currently, a most popularly used HLA typing method employs PCR amplification of a target region by designing primers flanking an HLA allele, followed by DNA sequencing (for example, Patent Document 1). However, with this method, it is difficult to accurately estimate HLA alleles, because of problems such as amplification of pseudogene region due to an effect from off-target amplification when using the amplification primers. Although Patent Document 1 aims to solve this problem by using a specific primer set, it is likely that there will be a certain amount of inaccuracy in HLA typing as it uses PCR amplification, because the target region of the HLA locus is practically limited, and there exists wide variety of polymorphisms between individuals in HLA genes.

Furthermore, there is a method to determine HLA types by SNP genotyping at an HLA locus by designing cDNA probes based on known HLA allele sequences (Itoh Y et al., Immunogenetics 2005). However, with this method, it is difficult to determine new alleles, and there is a problem that individual genomic variation in alleles affects SNP typing accuracy.

### PRIOR ART DOCUMENTS

### Patent Documents

Patent Document 1: WO 2014/065410 A1

### DISCLOSURE OF THE INVENTION

### Problems to be Solved by the Invention

As described above, when determining genotypes in a particular locus of MHC by using high performance sequencers in any form, inaccuracy of amplification such as amplification of an off-target region, at the stage of performing a gene amplification method such as a PCR, and others, is always a problem regardless of how an appropriate amplification primer is used. There exists a similar problem, for any gene loci, where there are other loci with similar nucleotide sequences, or genetic polymorphism exists, during the process of determining genotypes of each locus of the gene loci.

Therefore, means for optimizing read information obtained by a high-performance sequencer that is aligned to a particularly complicated gene loci, such as MHC loci, is required for genotyping each locus.

The present invention aims to provide the optimizing scheme from the viewpoint of probabilistic statistical processing.

### Means for Solving the Problems

The inventors of the present invention selected HLA loci, which is human MHC loci, as a loci in the process of investigating toward solving this problem, and from the subject's sample DNA, whole-genome sequencing read data was obtained by a high-throughput sequencer, and then this read data was aligned to the HLA allele reference sequences of these loci to obtain mapping correspondence information between read data and HLA allele reference sequences. By performing maximum likelihood estimation procedure, or more preferably Bayesian inference procedure, the expected number of mappings of each read to each HLA allele, and the allele frequency of each HLA allele with respect to the read data were obtained, and the inventors have found that optimization of the expected number of mappings for each HLA allele that can be used for extremely accurate HLA typing is done, and the inventors completed the present invention which can accurately determine the allele at each locus where there are multiple loci having similar nucleotide base sequences in the genome or a large number of genetic polymorphisms exits in these loci. In the present invention, the “high-throughput sequencer” is a sequencer that can provide a large amount of read information in a relatively short period of time, which can be used in the practice of the present invention, and includes a so-called “next generation sequencer”. Examples of the next generation sequencer at the time of carrying out the present invention include Genome Sequencer FLX (Roche (454)), Genome Analyzer IIx, HiSeq 2000, HiSeq 2500, MiSeq (both Illumina), SOLiD (Applied Biosystems), PacBio RS II (Pacific Biosciences) and the like. However, the high-throughput sequencer in the present invention is not limited to these sequencers, and includes all the high-throughput sequencers currently provided, and will be provided in the future.

At the time of completion of the present invention, two types of read information are generally recognized: single-end read information and paired-end read information. The single-end read information is read information on a fixed length or variable length (about 50 to 300 bp in general) at one end of the base sequence of the DNA fragment corresponding to the read, and the paired-end read information is read information on a fixed length or variable length (about 50 to 300 bp in general) at both ends of the DNA fragment. Although the content of the read information is also progressing rapidly in accordance with the advancement of the technology, it is possible to apply the present invention to the present or future read information.

As described above, the selected gene loci that include a gene locus where there are multiple genes or pseudogenes having a similar nucleotide sequence, and gene loci where there are many known genetic polymorphisms (alleles) exist at each locus, are the preferred loci. For such gene loci, as a common general knowledge of those skilled in this field, for example, cytochrome P450 (Cytochrome P450: CYP) loci, gene loci encoding immunoglobulin, gene loci encoding T cell receptors, gene loci encoding olfactory receptors, and the like are known, in addition to the MHC loci such as HLA loci. Cytochrome P 450 is a generic term for enzymes belonging to the oxidoreductase family involved in drug metabolism and detoxification, and 57 functional genes and 58 pseudogenes are known in human. Furthermore, at least 2,000 genetic polymorphisms (alleles) of the loci have been known so far, and it is known that metabolic rate of various drugs for each individual is different according to these genetic polymorphisms (alleles). Even if the HLA loci, which is one of the MHC loci of the examples disclosed in the present specification, is replaced by another complex loci, for example, the cytochrome P450 loci, it was obvious at the time of completion of the present invention that similar convenient utilities are obtained.

In addition, an individual gene locus is a gene locus constituting the gene loci, and for example, each of HLA-A, HLA-B, HLA-C is an individual gene locus for HLA gene loci.

### [A] The Optimization Method of the Present Invention

The present invention provides a method for optimizing read information of DNA (hereinafter also referred to as the optimization method of the present invention), which is characterized by executing all or a part of the following steps (1) to (6) using read information in which mapping correspondence of each read to alleles of particular gene loci or an individual gene locus (hereinafter also referred to as read mapping information of particular gene loci or an individual gene locus) is obtained by mapping a nucleotide sequence of a DNA read of data where read information of DNA derived from alleles of selected gene loci or an individual gene locus (hereinafter also referred to as particular gene loci or an individual gene locus) is mixed. The optimization method of the present invention is a method executed in a computer.
(1) A step in which, for each read, quantification of the expected number of mappings to each allele of particular gene loci or an individual gene locus, is performed.

“Expected number of mappings” is defined for each read against each allele, “total number of expected mappings” described later is defined for each allele, and “sum of total number of expected mappings” is the number of mappings defined for the gene loci or individual gene locus. In the present invention, “mapping” and “alignment” are synonymous.
(2) A step in which the expected number of mappings quantified in step (1) is summed for each allele of the gene loci or individual gene locus to calculate the total number of expected mappings.

The step (2) can be represented by a mathematical expression, for example, by the following formula (I), showing derivation of the total number of expected mapping rₜ generated from the allele t of particular gene loci or an individual gene locus by summing the currently estimated values of the abundance parameter.

[Mathematical 1]

rₜ−ΣₙE_{z}[Z_{n t}] (I)
[In the expression, Zₙₜ is an indicator variable, where it equals to 1, if read n is generated from the allele t, and 0 otherwise, and E_{z}[Zₙₜ] is an expected value of Zₙₜ. Here, an expected value of Zₙₜ is equivalent to the posterior probability of Zₙₜ=1.]
(3) A step that calculates a fraction of reads allocated to each allele of the gene loci or individual gene locus relative to the total read amount mapped to alleles of the gene loci or individual gene locus, where the total number of expected mappings calculated in step (2) is divided by the sum of the total number of expected mappings of all the alleles of the gene loci or individual gene locus.

The step (3) can be represented by a mathematical expression, for example, by the following expression (II), showing E_{q}[θₜ], a fraction of reads assigned to an allele of particular gene loci or an individual locus, relative to the total read amount.

[Mathematical 2]

E_{q}[θₜ]=rₜ/(Σ_{t′}r_{t′}) (II)
[In the expression, rₜ is the total number of expected mapping generated from the allele t of particular gene locus or an individual gene locus and θ is the parameter indicating the frequency of the abundance of reads on the allele of particular gene loci or an individual gene locus.]
(4) A step in which the fraction of reads obtained in step (3) is assigned to each allele of the gene loci or individual gene locus as frequency, and based on the assigned frequencies, for each read, the expected number of mappings to each allele of the gene loci or individual gene locus is calculated again.
(5) A step in which step (2) or (3) is executed again for calculating a fraction of reads allocated to an allele of the gene loci or individual gene locus relative to the total read amount mapped to alleles of the gene loci or individual gene locus, based on the updated expected number of mappings obtained in step (4).
(6) A step in which step (4) and (5) are repeatedly executed, until for all reads difference between the expected number of mappings to each allele of the gene loci or individual gene locus calculated in step (4) and that calculated in the previous step (4) is not recognized, or for all alleles in the gene loci or individual gene locus difference between the fraction of reads calculated in step (5) and that calculated in the previous step (5) is not recognized, and the converged expected number of mappings for each read for each allele of the gene loci or individual gene locus, or, the converged fraction of reads for alleles of the gene loci or individual gene locus, is recognized as optimized data.

In the optimization method of the present invention as described above, when for each read, the expected number of mappings for each allele of the particular gene loci or individual gene locus is estimated in steps (1) and (4), and the allele frequencies for each allele of the particular gene loci or individual gene locus are calculated in steps (3) and (5), an Expectation Maximization algorithm (EM algorithm) is used to perform maximum likelihood estimation, and more preferably, a variational Bayesian method is used to perform Bayesian estimation.

Here, assumptions for performing optimization means such as the EM algorithm and the variational Bayesian method will be described. This is a more detailed explanation of the disclosure in the earlier application (Japanese Patent Application No. 2014-265704: hereinafter also referred to as the earlier application) which is the basis of priority claim of the present application. In the present invention, besides the EM algorithm and the variational Bayes method, a Bayesian method, a Gibbs sampling method, a MCMC method, an EP method, a PowerEP method, and others can be used.

The symbols indicating parameters, numbers, and other variables described in the specification, claims, and drawings are symbols for convenience of disclosure of the invention, and the present invention is not limited by these types of symbols at all.

As variables and parameters to be estimated by an inference method of the present invention, observed data, objective parameters, and latent variables connecting observed data and objective parameters can be enumerated, and, for example, can be set as follows.

### [Observed Data]

Observed data (hereinafter also referred to as Rₙ) is “nucleotide sequence of a DNA read (hereinafter also referred to as read n) of data where read information of DNA derived from alleles of particular gene loci or an individual gene locus is mixed”, as described above. DNA mixed data is data as a whole, obtained by mapping reads to allele reference sequences of particular gene loci or an individual gene locus such as HLA loci or locus, HLA being a human MHC, as well as DNA reads themselves generated from DNA sequencing of a specimen. For example, when the particular gene loci is HLA loci, the reference sequence can be obtained from the IMGT/HLA database or other similar databases, but for these gene loci, genome sequences determined in the past studies using another specimen by sequencing or other methods is also possible to use as reference sequences. When a new genotype of the particular gene loci, for example, a new HLA type is found, it is preferable that the new genotype is successively registered into a database or other resources. It is also preferable that a novel genotype revealed by the present invention is registered. Determination of a new genotype with the present invention will be described later.

The observed data Rₙ is the nucleotide sequence of the n-th read data of the DNA mixed data of N (N is a natural number: the number of reads) as described above. It is assumed that read data is observed as N independent and uniformly distributed read data. In the case of single-end read, one observed data Rₙ corresponds to one read, but in the case of paired-end read, two observed data corresponds to a pair of nucleotide sequences at both ends of one fragment as a group. That is, in the case of a paired-end read, for example, a pair of Rₙₐ and R_{nb} is constructed, but since these are nucleotide information derived from the same fragment, by treating this set of reads as one unit, it is possible to handle it under the same statistical model similarly for single-end read (Nariai et al., Bioinformatics: 15; 29 (18) 2013). Specifically, it is possible to deal with the calculation as described in [Complete likelihood of paired-end model] described below based on the previous literature.

### [Objective Parameter]

The objective parameter is a parameter that is estimated on the basis of the observed data Rₙ. The present invention estimates one objective parameter (hereinafter also referred to as θ).

The objective parameter θ is a vector representing the allele frequency of particular gene loci or an individual gene locus such as HLA loci or locus, HLA being a human MHC loci. For example, the parameter vector θ=(θ₁, . . . , θ_{T})′ (hereinafter, “′” in a vector or matrix indicates a transpose unless otherwise specified), and the fraction of the abundance for each allele of particular gene loci or an individual gene locus can be expressed under the condition below.

Σₜ₌₁^{t=T}θₜ=1 [Mathematical 3]

In this case, it is assumed that there are T (T is a natural number) alleles of a particular gene loci or an individual locus exist, and an allele of particular gene loci or an individual locus is counted as t (t is an integer of 1 or more).

By estimating the objective parameter θ, it is possible to estimate the allele frequency or genotype of particular gene loci or an individual gene locus, which is the purpose of the present invention.

### [Latent Variables]

Latent variables, or hidden variables, are used to describe from which allele of particular gene loci or an individual locus the observed data Rₙ is generated, and from which position in an allele of particular gene loci or an individual gene locus it is generated. In the present invention, by estimating parameter θ with incorporating the two kinds of latent variables (Tₙ, Sₙ) or Tₙ alone, these objective variables can be accurately estimated, and further, it is possible to estimate the genotype of each locus of the particular gene loci or the individual gene locus such as HLA loci or locus, HLA being a human MHC. It is possible to precisely estimate the genotype of each locus by estimating the parameter θ from the observed data Rₙ, where these latent variables are incorporated as dependent on the observed data Rₙ.

The above latent variable Tₙ is a variable depending on the above θ, representing allele selection of particular gene loci or an individual gene locus such as HLA loci or locus, HLA being a human MHC, of read n. Tₙ=t means that read n is generated from allele t of particular gene loci or an individual gene locus.

The above latent variable Sₙ is a variable depending on Tₙ representing the start position of the read n. Sₙ=s indicates that read n is generated from position s (1≦s≦lₜ−L+1) (where lₜ is the length of allele t of particular gene loci or an individual gene locus and L is the read length). Here, the length lₜ of the allele of particular gene loci or an individual gene locus such as HLA loci or locus, HLA being a human MHC, which is a target of the present invention, is several hundreds of bases to several tens of thousands of bases in length, and generally, the length is longer than the base length of the read. Also, if the starting position s is 1, it means that the read has been generated from the first base of the allele of the particular gene loci or individual locus. In other words, Sₙ means the starting position in the reference sequence of the allele of the particular gene loci or individual locus such as HLA loci or locus, HLA being human MHC, when the read is mapped to the reference sequence.

As described later, in particular in the case of a paired-end read model, for example, a latent variable Fₙ and others representing the length of DNA fragment between the paired-end reads can be incorporated into genotype estimation, together with Tₙ and Sₙ.

### [Representation of the Estimation Method of the Present Invention]

For example, the estimation method of the present invention using the above variables can be represented as “An optimization method in which mapping of the read information of the DNA from the subject to alleles of particular gene loci or an individual gene locus is optimized by a computer, including a step to determine for each read the expected number of mappings to alleles of the gene loci or individual gene locus, and a step to estimate allele frequencies θ of the gene loci or individual gene locus as an object parameter (where θ is the T dimensional vector, T is the number of alleles of the gene loci or individual gene locus), given nucleotide sequences of all reads in data in which read information of DNA derived from alleles of selected gene loci or an individual gene locus is mixed, as observed data R, that is characterized in that with respect to (a) a latent variable Tₙ dependent on θ related to the allele selection of the gene loci or individual gene locus of read n, and (b) a latent variable Sₙ dependent on Tₙ related to the starting position of n, and given the nucleotide sequence of read n as observed data Rₙ, at least (i) variables Tₙ and Sₙ or (ii) variable Tₙ is incorporated to calculate an estimate of the parameter in the estimation process of the objective parameter θ, so that observed data Rₙ depends on the latent variables during the estimation process of the objective parameter θ from observed data Rₙ”.

### [Complete Likelihood of Single-End Model]

The complete likelihood (posterior joint distribution) of the parameters of the optimization method of the present invention including dependency between variables using the above variables is decomposed as the product of the conditional probabilities. Specifically, it is represented by the following formula (1). Each symbol is as described above unless otherwise specified.

[Mathematical 4]

p(Tₙ, Sₙ, Rₙ|θ)=p(Tₙ|θ) p(Sₙ|Tₙ) p(Rₙ|Tₙ, Sₙ) (1)

In the formula (1), p(Tₙ=t|θ) is the probability that read n is generated from allele t of particular gene loci or an individual locus given θ. This probability can be calculated as p(Tₙ=t|θ)=θₜ ((1) (a)).

p(Sₙ=s|Tₙ=t) is the probability that read n is generated from position s given the allele t of the particular gene loci or individual locus. This probability can be calculated as p(Sₙ=s|Tₙ=t)=1/(lₜ−L+1) ((1) (b)). lₜ is the length of the reference sequence of allele t, and L is the read length.

p(Rₙ|Tₙ=t, Sₙ=s) is the probability of observing the nucleotide sequence of read n, given allele selection of the particular gene loci or individual locus and, the starting position of read n. Here, it is preferable to introduce the indicator variable Zₙₜₛ for summarizing Tₙ and Sₙ, or the indicator variable Zₙₜ for summarizing Tₙ ((1) (c)).

Zₙₜₛ is equal to 1 when (Tₙ, Sₙ)=(t, s), and is zero otherwise. Suppose πₙ is a set of all (t, s) pairs for possible mappings of read n, then for each (t, s) ∈ πₙ the following equation (2) can be used to calculate the probability of observing read sequence:

$$\begin{matrix}[ Mathematical 5 ] & \\ p ( {R}_{n} \vert {Z}_{nts} = 1 ) = {\prod }_{x = 1}^{L} subst ( {r}_{n} [ x ] , {q}_{n} [ x ] , {c}_{t} [ x ] ) & ( 2 )\end{matrix}$$

(where subst(,,) is a base quality score dependent substitution probability function taking a numerical value obtained by subtracting a sequence substitution error from 1, rₙ[x] is the nucleotide character at position x of read n, qₙ[x] is the base quality score of position x of read n, and cₜ[x] is the base character of position x of the corresponding DNA sequence of allele t of the particular gene loci or individual locus.)

The base quality score substitution probability function, “subst(,,)” can be determined according to the Phred base quality score and can also be estimated from the best alignment of the read to the reference DNA sequence from the DNA-Seq data. The Phred base quality score is a score indicating a base reading accuracy provided together with nucleotide sequence information outputted in FASTQ format from a high-throughput sequencer, that is, a score indicating an error rate outputted by the sequencer (Phred quality Score). Specifically, the score Q is calculated as follows:

Q=−10 log₁₀ Y (Y is the error rate).

On the other hand, Zₙₜ is equal to 1 if Tₙ=t, and is zero otherwise. Since Zₙₜ takes all the Zₙₜₛ mentioned above into consideration with respect to possible s for each t, from (2), the probability of observing read sequence can be calculated as below.

$$\begin{matrix}[ Mathematical 6 ] & \\ p ( {R}_{n} \vert {Z}_{nt} = 1 ) = {\sum }_{s = 1}^{{I}_{t} - L + 1} p ( {R}_{n} \vert {Z}_{nts} = 1 ) & {( 2 )}^{′}\end{matrix}$$

Although the above is a calculation formula that takes into consideration the sequence substitution errors, equations that take into consideration sequence insertion/deletion errors can also be easily derived (Nariai et al., Bioinformatics: 15; 29 (18)).

The complete likelihood formula of the estimation method of the present invention shown in the above formula (1) is interpreted as described above.

This equation (1) serves as the basis for finding the posterior probability and posterior distribution of the latent variable according to the estimation method of the present invention using single-end model.

As for the latent variable Zₙₜ, it is possible to define it as independent of Zₙₜₛ, that is, not as a latent variable based on Zₙₜₛ as described above, in which the mapped position “s” described above is not considered at all. For example, if the read n is mapped somewhere in the allele t, it is also possible to calculate the expected number of mappings using only the mapping score given by the mapping tool without considering the position “s”. In principle, latent variable Zₙₜ in the following disclosure is used as a variable based on Zₙₜₛ, but it is also possible to use Zₙₜ as a variable independent of Zₙₜₛ shown here.

### [Complete Likelihood of Paired-End Model]

In the case of paired-end data, complete likelihood (posterior joint distribution) of the parameters of the optimization method of the present invention including dependency between variables using the above variables is decomposed as the product of the conditional probabilities. Specifically, it is represented by the following expression (3). Each symbol is as described above unless otherwise specified.

[Mathematical 7]

p(Tₙ,Sₙ,Fₙ,Rₙₐ,R_{nb}|θ)=p(Tₙ|θ)p(Fₙ|Tₙ)p(Sₙ|Tₙ,Fₙ)p(Rₙₐ|Tₙ,Sₙ)p(R_{nb}|Tₙ,Sₙ,Fₙ) (3)

Here, Fₙ is the length of a nucleotide fragment (fragment) estimated from the mapping of the pair of paired-end reads “Rₙₐ and R_{nb}” to the reference sequence.

On the right-hand side of expression (3), p(Tₙ=t|θ) is the probability that read n is generated from allele t of particular gene loci or an individual gene locus given θ. This probability can be calculated as p(Tₙ=t|θ)=θₜ ((3) (a)).

p(Fₙ=f|Tₙ=t) is the probability that the length f of the nucleotide fragment will be generated given the allele t of the locus. Let d_{F}(x) be the distribution of the length of the nucleotide fragment given in advance. The probability can be calculated as below.

[Mathematical 8]

p(Fₙ=f|Tₙ=t)=d_{F}(f)/Σ_{x=L}^{l}^{ₜ}d_{F}(x) (3)(a′)

Here lₜ is the length of the reference sequence of allele t, and L is the read length. Distribution d_{F}(x) of the length of the nucleotide fragment is given as a normal distribution of, for example, mean μ_{F} and standard deviation σ_{F}. The mean μ_{F} and the standard deviation σ_{F} may be specified as long as the distribution of the nucleotide fragment length is experimentally known at the time of preparing the nucleotide fragment, but can be estimated and specified from the result of alignment of many paired-end reads beforehand.

p(Sₙ=s|Tₙ=t, Fₙ=f) is the probability that the pair of read n of fragment length f is generated from position s, given the allele t of the particular gene loci or individual gene locus. This probability can be calculated as p(Sₙ=s|Tₙ=t, Fₙ=f)=1/(lₜ−f+1) ((3) (b)). lₜ is the length of the reference sequence of allele t, L is the lead length, and f is the length of the base fragment.

p(Rₙₐ|tₙ=t, Sₙ=s) and p(R_{nb}|Tₙ=t, Sₙ=s, Fₙ=f) can be calculated given allele selection of the particular gene loci or individual gene locus, start position of the pair of read n, and fragment length by the following expressions (4-1, 4-2):

$$\begin{matrix}[ Mathematical 9 ] & \\ p ( {R}_{na} \vert {T}_{n} , {S}_{n} ) = {\prod }_{x = 1}^{L} subst ( {r}_{na} [ x ] , {q}_{na} [ x ] , {c}_{ta} [ x ] ) & ( 4 \text{-} 1 ) \\ p ( {R}_{nb} \vert {T}_{n} , {S}_{n} , {F}_{n} ) = {\prod }_{x = 1}^{L} subst ( {r}_{nb} [ x ] , {q}_{nb} [ x ] , {c}_{tb} [ x ] ) & ( 4 \text{-} 2 )\end{matrix}$$

Here, subst(,,) is a base quality score dependent substitution probability function taking a numerical value obtained by subtracting a sequence substitution error from 1, rₙₐ[x] is nucleotide base character of position x of the first pair of read n (Rₙₐ), qₙₐ[x] is the base quality score of position x of the first pair of read n, cₜₐ[x] is the corresponding nucleotide base character of allele t of the particular gene loci or individual gene locus at which the first pair of read n is aligned at position x, r_{nb}[x] is nucleotide base character of position x of the second pair of read n (R_{nb}), q_{nb}[x] is the base quality score of position x of the second pair of read n, and c_{tb}[x] is the corresponding nucleotide base character of allele t of the particular gene loci or individual gene locus at which the second pair of read n is aligned at position x.

Expression (3) above is a basis for calculating the posterior probability and posterior distribution of the latent variables according to the estimation method of the present invention using the paired-end model.

The disclosure of the latent variable Zₙₜ in the above single-end model can also be applied to this paired-end model.

### [Hyperparameter]

In particular, when Bayesian estimation method such as variational Bayesian method is performed as the estimation means, it is preferable that hyperparameter α₀ (0<α₀) is incorporated and calculated, especially 0<α₀≦0.1, or α₀ that maximizes the lower bound of the log likelihood being preferred. It is possible to carry out Bayesian estimation that is robust against outliers by incorporating an appropriate hyperparameter α₀ of an appropriate value.

This can be expressed, for example, by the following formula (II)′ based on the above formula (II).

[Mathematical 10]

E_{q}[θₜ]=αₜ/(Σ_{t′}α_{t′}) (II)′

[where αₜ=α₀+rₜ]

Hyper parameter α₀ is a constant to be considered in the framework of Bayesian estimation. That is, the parameter θ indicating the abundance of reads on alleles of the particular gene loci or individual gene locus can be estimated as a posterior distribution in the framework of Bayesian estimation, and here, we assume the Dirichlet distribution (Formula (III)):

$$\begin{matrix}[ Mathematical 11 ] & \\ p ( θ ) = \frac{1}{C} {\prod }_{t = 1}^{T} {θ}_{t}^{{α}_{t} - 1} & ( III )\end{matrix}$$

[where C is a constant, Π^{T}ₜ₌₁θₜ=1, T is the number of alleles at particular gene loci or an individual gene locus under consideration, and αₜ is the hyperparameter.]

The hyperparameter α₀ which controls the complexity of the parameter θ (the number of θₜ>0) is selected such that it maximizes the log marginal likelihood of the observed data.

Estimating the posterior distribution of θ given observed data requires integral with respect to latent variables and is difficult to calculate in closed form. Therefore, by assuming the factorization of the latent variables and the parameter θ, the equation is derived by approximating integrals in the posteriori probability distribution, which is described in formula (II)′.

The hyperparameter will be described later.

The optimization method of the present invention described above can be used without any particular limitation as long as it is applied to the read mapping information of the particular gene loci or individual gene locus. For example, the read mapping information of the gene locus can be the one obtained by processing with a high-throughput sequencer on a gene amplification product prepared with a primer corresponding to a certain locus of the particular gene loci or individual gene locus, or alternatively, it can be read information obtained by mapping the read mapping information of the gene locus to alleles at the locus. However, the optimization method of the present invention can be applied to the read mapping information of particular gene loci or an individual gene locus obtained by mapping read information generated from whole-genome sequencing of the subject sample to alleles of the particular gene loci or individual gene locus, without performing the step of amplifying genetic elements of the subject at a gene locus of the particular gene loci or individual gene locus.

In the present invention, an optimization method of the present invention is provided, which is characterized by executing the following steps (a) and (b) in mapping reads for acquiring mapping read information of the particular gene loci or individual gene locus. Hereinafter, the process of performing these steps is also referred to as a “mapping process of the particular gene loci or individual gene locus of the present invention”.
(a) A step in which reads are mapped to the human reference genome sequence and then those mapped to alleles of particular gene loci or an individual gene locus are extracted, where the reads contain nucleotide sequence information of a subject generated from a DNA sequencer.

It is preferred that this initial mapping target is a human whole genome sequence, and it is customary that the target is the genome sequence of a specific person (a person selected as an analysis target or a combination of specific persons). It is usually a genome sequence determined by the Genome Reference Consortium or other institutions. With this first mapping, reads unrelated to alleles of the particular gene loci or individual gene locus can be excluded from the downstream analysis. In addition to the human whole genome sequence described above, for example, targeted sequence data, Exome sequence data, RNA sequence data, and long-read sequence data such as PacBio RS II, Oxford Nanopore and others can also be used as the mapping target sequence.
(b) A step in which the read sequence information mapped to alleles of the particular gene loci or individual gene locus obtained in step (a) is mapped to reference nucleotide sequences of alleles of the particular gene loci or individual gene locus that are registered in a database, and reads mapped to each allele of the gene loci or individual gene locus are extracted, and read information is obtained, in which mapping of each read to each allele of the gene loci or individual gene locus is identified.

The target of this second mapping is the genomic sequence of all alleles of the particular gene loci or individual gene locus registered in a database. As a result, it is possible to obtain provisional read mapping information of the particular gene loci or individual gene locus.

The mapping process in step (b) above, which corresponds to the mapping process of the particular gene loci or individual gene locus of the present invention, is preferred to be executed with allowing multiple mapping to alleles of the particular gene loci or individual gene locus. When the mapping targets are intentionally narrowed down at this point, there is a strong possibility that inappropriate biases will be included with respect to the read mapping information of the particular gene loci and individual gene locus to be derived.

As described above, the sequence information of reads obtained by a DNA sequencer used in the above step (a) may be the sequence information of paired-end, in which reads from both ends of each DNA fragment (about 50 to 300 bp each) are generated. Although the sequence information of single-end, in which reads from one side of each DNA fragment (about 50 to 300 bp) may be used, but paired-end read data is desirable to achieve higher accurate mapping, and as a result, higher accurate estimation of alleles, because the sequence information corresponding to one DNA fragment (usually about 300 to 1000 bp) can be specified based on the range bounded by the reads of both ends.

Also, it is preferable that, in addition to the reads mapped to alleles of the particular gene loci or individual gene locus in step (a), reads not mapped to the human genome are also extracted and are used in step (b). This is because the target genome for the mapping in the above step (a) is a genome of a specific person or a combination of multiple persons, and it is generally assumed that genome sequences will not be the same compared with that of the sample. For example, when the particular gene loci is HLA loci, HLA being human MHC, if the target genome for the mapping is the genome of Westerners and the subject is Japanese, then there is a possibility that the sequence of HLA loci differs greatly from the target genome, and hence there is a possibility that reads derived from HLA loci of the subject is not mapped to the target genome for mapping. In order to avoid this problem, the above processing is performed.

### [B] The Determination Method of the Present Invention

It is possible to use the read mapping information of the particular gene loci or individual gene locus obtained by the optimization method of the present invention for an indicator of the genotype of each locus of the particular gene loci or individual gene locus. Particularly, if reads are generated from alleles of the gene locus by gene amplification operation such as PCR method using a gene amplification primer corresponding to the particular gene loci or individual gene locus at the stage of using the high-throughput sequencer, this tendency is easily recognized. In that case, the individual depth of coverage of reads for each allele of the gene locus is calculated from the fraction of reads in the read mapping information of the gene locus, and one or two alleles with the largest individual depth of coverage are determined as the genotype of the gene locus (determination method of the present invention). However, even in such cases, it is preferable to reexamine the results and eliminate the possibility of false positives as much as possible.

In addition, when the processes that narrow down to the gene locus of the gene loci using the gene amplification method was not performed beforehand, including the case where the mapping process of the particular gene loci or the individual gene locus of the present invention is performed, it is more preferable to reexamine the results.

In the case of a method that does not narrow down using a gene amplification method beforehand (for example, when a whole-genome reference sequence is used as a reference sequence), it is more desirable to perform the following reexamination process.

A preferred embodiment of the determination method of the present invention is a method of determining the genotype of each locus of the particular gene loci or individual gene locus extremely accurately, by re-evaluating read mapping information of the particular gene loci or individual gene locus obtained by the optimization method of the present invention described above with calculation of the individual depth of coverage.

Thus, the present invention provides genotype determination method for each locus of the particular gene loci or individual gene locus, which is characterized by calculating the individual depth of coverage of each allele from the fraction of reads on the allele of the gene loci or individual gene locus obtained by the optimization method of the present invention, by selecting the top two or less than two alleles of a gene locus of the particular gene loci or individual gene locus from a list of alleles that are sorted based on the individual depth of coverage by descending order, by setting a threshold of depth of coverage of alleles at 5-50%, or more preferably, 10-30%, of the depth of coverage of total reads in the determination method of genotype of each locus of the particular gene loci and individual gene locus for determining alleles as the genotype of the gene locus, and by eliminating alleles of the particular gene loci or individual gene locus whose individual depth of coverage are less than the threshold from genotyping of the gene loci or individual gene locus.

The “depth of coverage of total reads” indicates the average number of duplicate reading performed on the genome of the subject with a high-throughput sequencer, calculated based on the number of DNA fragments at each nucleotide position. Specifically, it is the total number of nucleotide bases contained in all reads generated by the sequencer divided by the total number of nucleotide bases of the genome (total human genome is 3 billion nucleotide bases). For example, if there are 900 million reads of 100 bp single-end, the depth of coverage of total reads is “100×900 million/3 billion=30”, which is described as “30×”. Here, “total number of nucleotide bases of the genome” becomes the total number of nucleotide bases at a specific HLA locus, when this is not a whole-genome sequence data, for example, when the target region is “a particular HLA locus”.

The “individual depth of coverage” means, in the present invention, indicates on average how many reads are overlapped at each position of the allele in the particular gene loci or individual gene locus, which can be calculated from the fraction of reads on the allele of the particular gene loci or individual gene locus obtained by the optimization method of the present invention.

Specifically, “the number of reads allocated to the allele of the particular gene loci or individual gene locus” is defined as “the fraction of reads on the allele of the particular gene loci or individual gene locus×total number of reads mapped to alleles of the gene loci or individual gene locus”.

As described above, “the fraction of reads on the allele of the particular gene loci or individual gene locus” is calculated according to the optimization method of the present invention. Also, “the total number of reads mapped to alleles of particular gene loci or individual gene locus” is derived in, for example, “mapping process of the particular gene loci or individual gene locus of the present invention”. Thus, “the number of reads allocated to the allele on the particular gene loci or individual gene locus” is calculated by performing the optimization method of the present invention.

Here, “individual depth of coverage” is calculated by “total number of nucleotide bases of reads mapped to the allele of particular gene loci or individual gene locus/the number of nucleotide bases of the allele reference sequence of the gene loci or individual gene locus”.

Since an average number of nucleotide bases per read is known, “the total number of nucleotide bases of reads mapped to the allele at the particular gene loci or individual gene locus” is calculated by multiplying “the number of reads allocated to the allele at the gene locus” by the average number of nucleotide bases per read. “The number of nucleotide bases of allele reference sequence of the particular gene loci or individual gene locus” is a known number, which is different for each allele of the particular gene loci or individual gene locus. Thus, the “individual depth of coverage” of the allele of the particular gene loci or individual gene locus is, as described above, calculated based on the fraction of reads on the allele of the particular gene loci or individual gene locus obtained by the optimization method of the present invention.

In summary, the individual depth of coverage “dₜ” in the allele of the particular gene loci or individual gene locus is calculated by the following equation (IV).

$$\begin{matrix}[ Mathematical 12 ] & \\ {d}_{t} ( {\sum }_{n = 1}^{N} E [ {Z}_{nt} ] {c}_{n} ) / {l}_{t} & ( IV )\end{matrix}$$

[where N is the total number of reads, cₙ is the number of nucleotide bases contained in read n, E[Zₙₜ] is the expected number of mappings of read n to allele t at the locus, and lₜ is the length of reference sequence of allele at each locus (the number of nucleotide base). t can range from 1 to T (the total number of alleles at the locus), and n can range from 1 to N.]

Note that E[Zₙₜ] shown here can also be calculated by adding E[Zₙₜₛ] mentioned above with respect to all possible s for each allele t.

By setting the “rejection depth of coverage” based on the depth of coverage of total reads as described above, an allele of the particular gene loci or individual gene locus having a small individual depth of coverage is excluded from genotyping, which leads to increase in accuracy of the determination method of the present invention by excluding, so to speak, a false positive allele candidate of the particular gene loci or individual gene locus.

The rejection depth of coverage can be selected as the frequency number of 5 to 50%, preferably 10 to 30% of the depth of coverage of total reads as described above. If the rejection depth of coverage is small, alleles of the particular gene loci or individual gene locus have a greater chance of becoming candidates for genotyping of the particular gene loci or individual gene locus in the determination method of the present invention, but the risk of choosing false positives will also increase. Conversely, if the rejection depth of coverage is large, there is less possibility of choosing false positives, but there is a higher possibility that it rejects alleles that represent true genotype of the particular gene loci or individual gene locus of the subject.

The reason of selecting “the top two or less than two alleles of a gene locus of the particular gene loci or individual gene locus from a list of alleles that are sorted based on the individual depth of coverage by descending order”, or the maximum value of the number of alleles at each locus of the particular gene loci or individual gene locus is set to “two”, is that the two different alleles at the locus will be determined as heterozygotes of these, and alleles of more than this number are excluded as noise. If the number is “one”, the gene locus will be determined as homozygous of the allele, or heterozygous of the allele and another that is unknown, and if the number is “zero”, the alleles of the corresponding locus will be determined as unknown.

More specifically about a manner of the determination method of the present invention, after excluding alleles for genotyping the particular gene loci or individual locus as described above, it is preferable that the following (i) or (ii) is determined.
(i) For the case that there is one allele that was selected for genotyping the gene locus of the particular gene loci or individual gene locus, if the individual depth of coverage of the allele is twice or more of the rejection threshold value described before, it is determined that the genotype is homozygous of the allele, and if it is less than twice of the rejection threshold, it is determined that the genotype is heterozygous of the allele, and
(ii) For the case that there are two alleles that were selected for genotyping the gene locus of the particular gene loci or individual gene locus, if the individual depth of coverage of the one whose individual depth of coverage is larger is less than twice of the smaller one, it is determined that the genotype is heterozygous of the two alleles, or if the individual depth of coverage of the one whose individual depth of coverage is larger is twice or more of the smaller one, it is determined that the genotype is homozygous of the allele whose individual depth of coverage is larger.

Performing the determination method of the present invention in this manner makes the determination of alleles of each locus of the particular gene loci or individual gene locus of the subject more accurately.

When the allele to be determined is novel, a known allele that is closest to the novel allele is determined first, and by recognizing differences between the known allele and the novel allele due to substitutions, insertions, deletions, and other variations of the nucleotide sequence, the nucleotide sequence of the novel allele can be determined. It is preferable that the nucleotide sequence of the novel allele is successively registered as a new allele in a target database or other databases.

Hereinafter, the optimization method and determination method of the present invention may be collectively referred to as “the method of the present invention”.

### [C] The Computer System of the Present Invention

The computer system of the present invention is a system which is a device for performing the method of the present invention described above, and the same terms overlap as a concept unless otherwise noted. “Algorithm” means a formalized form of a procedure for solving a problem, which is the same as a general concept in computer fields.

The computer system of the present invention can include hardware related to a normal computer system. That is, in addition to a “recording unit” corresponding to a normal hard disk drive and an “arithmetic processing unit” corresponding to a CPU, for example, a “temporary storage unit” corresponding to RAM, an “operation unit” corresponding to a keyboard, a mouse, a touch panel, and other interfaces, a “display unit” corresponding to a display, an “input/output interface (IF) unit” corresponding to a serial or parallel interface corresponding to an operation unit, and a “communication interface (IF) unit” equipped with a video memory and a D/A conversion unit for outputting analog signal corresponding to the video mode of the display unit. The communication IF unit can exchange data with external information, particularly human genome information such as human genome databases.

Unless stated otherwise, the processing performed by the “processing unit” of the computer system of the present invention will be described below. “Operation processing unit” acquires data of the human genome database, in particular via the “communication IF unit” by operating the “operation unit”, records it in the “recording unit”, stores data read from the “recording unit” to the “temporary storage unit” occasionally, performs predetermined processing, and records the result again in the “recording unit”. The “processing unit” creates screen data for prompting the operation of the “operation unit” and screen data for displaying the processing result, and displays these images on the “display unit” via the video RAM of the input IF unit. The program of the present invention is recorded at the time of use or recorded in advance in the “recording unit” or recorded in external hardware resources, and in “arithmetic processing unit” as necessary, arithmetic processing according to the described algorithm is performed.

The computer system of the present invention is a computer system for optimizing the read mapping information of the particular gene loci or individual locus, comprising a recording unit and an arithmetic processing unit, which is characterized in that all or a part of the following processes (A) to (G), is executed:
(A) In the recording unit, read information of DNA derived from the subject is recorded as data of nucleotide sequence of read and alleles of the gene loci or individual gene locus to which the read is mapped to,
(B) In the arithmetic processing unit, based on the information of the recording section, for each read, the expected number of mappings for each allele of the gene loci or individual gene locus is calculated by executing numerical processing,
(C) For each allele, the expected number of mappings quantified in the process (B) is summed for each allele of the gene loci or individual gene locus to calculate the total number of expected mappings,
(D) For each allele, the total number of expected mappings calculated in the process (C) is divided by the sum of the total number of expected mappings for all alleles of the gene loci or individual gene locus, and the process of calculating the fraction of reads allocated to each allele of the gene loci or the individual gene locus with respect to the total amount of reads mapped to alleles of the gene loci or individual gene locus is executed,
(E) The fraction of reads calculated in the above process (C) is assigned as frequency to each allele of the gene loci or individual gene locus, and given the assignment frequency, the process (B) described above is executed again in which for each read, the expected number of mappings to each allele of the gene loci or individual gene locus is calculated,
(F) The process (C) or (D) is executed again for the newly obtained expected number of mappings calculated by the process (E) described above, and the process of calculating the fraction of reads allocated to each allele of the gene loci or individual gene locus with respect to the total amount of reads mapped to alleles of the gene loci or the individual gene locus is executed,
(G) the processes (E) and (F) described above are repeatedly executed, until for all reads difference between the number of expected mappings to each allele of the gene loci or individual gene locus calculated in the process (E) and that calculated in the previous process (E) is not recognized, or for all alleles of the gene loci or individual gene locus difference between the value of the fraction of reads calculated in the process (F) and that calculated in the previous process (F) is not recognized, and the converged number of expected mappings for each read to each allele of the gene loci or individual gene locus, or the converged value of the fraction of reads for each allele (allele frequency) of the gene loci or individual gene locus, is recognized as the optimized data.

It is preferable that the above processes (B) to (G) are performed comprehensively on all alleles of the particular gene loci or individual gene locus. This comprehensive execution means that the optimization algorithm of mapping all reads is executed for all alleles of the gene loci or individual gene locus at the same time.

Furthermore, in the computer system of the present invention, the mapping of the read information of the gene of the subject to alleles of the gene loci or individual gene locus that are registered in the database can be executed by the following processes (a) and (b). In the case where the particular gene loci are targeted, it is preferable that the following processes (a) and (b) are carried out simultaneously for all the loci.
(a) The process in which after mapping sequence information of the subject's reads obtained by a DNA sequencer to the reference nucleotide sequence of whole human genome, reads mapped to each locus of the gene loci or individual gene locus are extracted.
(b) The process in which after mapping the reads mapped to the gene loci or individual gene locus extracted from the process (a) to nucleotide sequences of alleles of the gene loci or individual gene locus registered in the database, mapping information and mapping states, that is, identified read information of mapping position, differences between the read sequence and the reference sequence, and a mapping score, for each read to each allele of the gene loci or individual gene locus, are obtained.

It is preferred that the mapping performed in the process (b) allows one read to be mapped to multiple alleles of the particular gene loci or individual gene locus.

It is preferred that in addition to the reads mapped to alleles of the particular gene loci or individual gene locus obtained in the process (a), reads that were not mapped to the whole human genome are extracted and processed together, so that it becomes the target of the re-mapping in the process (b).

Furthermore, the present invention provides a computer system for determining the genotype of each gene locus of the particular gene loci or individual gene locus of a subject, comprising a recording unit and an arithmetic processing unit, and is characterized in that all or a part of the following processes (α) to (δ) is executed;
(α) In the recording unit, at least allele frequencies and depth of coverage of total reads of the gene loci or individual gene locus of the subject that were obtained by the optimization method of the present invention are recorded,
(β) In the arithmetic processing unit, based on the allele frequencies of the gene loci or the individual gene locus based on the recording unit, processing of calculating the individual depth of coverage of each allele of the gene loci or individual gene locus, and processing of allocating the calculated individual depth of coverage to the alleles of the gene loci or the individual gene locus, are executed,
(γ) Given a rejection threshold value, which is set as a frequency number of 5 to 50%, preferably 10 to 30% of the average depth of coverage of all reads, the process in which alleles of the gene loci or individual gene locus whose individual depth of coverage is smaller than the threshold value are excluded from candidates for genotyping,
(δ):
(δ)-1 After the exclusion process of (γ), for the case that there is one allele that was selected for genotyping the gene locus of the particular gene loci or individual gene locus, if the individual depth of coverage of the allele is twice or more of the rejection threshold value described before, it is determined that the genotype is homozygous of the allele, and if it is less than twice of the rejection threshold, it is determined that the genotype is heterozygous of the allele, and
(δ)-2 After the exclusion process of (γ), for the case that there are two alleles that were selected for genotyping the gene locus of the particular gene loci or individual gene locus, if the individual depth of coverage of the one whose individual depth of coverage is larger is less than twice of the smaller one, it is determined that the genotype is heterozygous of the two alleles, or if the individual depth of coverage of the one whose individual depth of coverage is larger is twice or more of the smaller one, it is determined that the genotype is homozygous of the allele whose individual depth of coverage is larger.

In addition to the above, for example, it is also possible to carry out the setting in the computer system of the present invention, in which for the case that there is no allele found for the genotype of each locus of the particular gene loci or individual gene locus after execution of the exclusion process of (γ), the genotype of the gene locus based on the allele is not determined.

The categories of these computer systems are “objects” and can be replaced with “devices”.

### [D] The Program of the Present Invention

The program of the present invention is a computer program implementing an algorithm run in the computer system of the present invention to execute the method of the present invention, and the same terms overlap as a concept unless otherwise noted.

The program of the present invention is a computer program for optimizing the read mapping information of the particular gene loci or individual gene locus, which is characterized by including algorithms to realize all or a part of the following functions (1) to (7):
(A) The function (1) in which read information of DNA derived from the subject recorded in the recording unit as data of nucleotide sequence of read and alleles of the gene loci or particular gene locus to which the read is mapped is retrieved,
(B) The function (2) in which based on the read information retrieved in the function (1), for each read, the expected number of mappings for each allele of the gene loci or individual gene locus is calculated by executing numerical processing,
(C) The function (3) in which for each allele, the expected number of mappings quantified in the function (2) is summed for each allele of the gene loci or individual gene locus to calculate the total number of expected mappings,
(D) The function (4) in which for each allele, the total number of expected mappings calculated in the function (3) is divided by the sum of the total number of expected mappings for all alleles of the gene loci or individual gene locus, and the process of calculating the fraction of reads allocated to each allele of the gene loci or the individual gene locus with respect to the total amount of reads mapped to alleles of the gene loci or individual gene locus is executed,
(E) The function (5) in which the fraction of reads calculated in the above function (4) is assigned as frequency to each allele of the gene loci or individual gene locus, and given the assignment frequency, the function (2) described above is executed again in which for each read, the expected number of mappings to each allele of the gene loci or individual gene locus is calculated,
(F) The function (6) in which the function (3) or (4) is executed again for the newly obtained expected number of mappings calculated by the function (5) described above, and the process of calculating the fraction of reads allocated to each allele of the gene loci or individual gene locus with respect to the total amount of reads mapped to alleles of the gene loci or the individual gene locus is executed,
(G) The function (7) in which the function (5) and (6) described above are repeatedly executed, until for all reads difference between the number of expected mappings to each allele of the gene loci or individual gene locus calculated in the function (5) and that calculated in the previous function (5) is not recognized, or for all alleles of the gene loci or individual gene locus difference between the value of the fraction of reads calculated in the function (6) and that calculated in the previous function (6) is not recognized, and the converged number of expected mappings for each read to each allele of the gene loci or individual gene locus, or the converged value of the fraction of reads for each allele of the gene loci or individual gene locus, is recognized as the optimized data.

Furthermore, the present invention provides the program of the present invention which is characterized by including algorithms of mapping of the read information of the gene of the subject to alleles of the particular gene loci or individual gene locus that are registered in the database by executing the following functions (a) and (b) to be realized in the computer.
(a) The function in which after mapping sequence information of the subject's reads obtained by a DNA sequencer to the reference nucleotide sequence of whole human genome, reads mapped to alleles of each locus of the gene loci or individual gene locus are extracted.
(b) The function in which after mapping the reads mapped to alleles of the gene loci or individual gene locus extracted from the function (a) to nucleotide sequences of alleles of the gene loci or individual gene locus registered in the database, mapping information and mapping states, that is, identified read information of mapping position, differences between the read sequence and the reference sequence, and a mapping score, for each read to each allele of the gene loci or individual gene locus, are obtained.

Furthermore, the present invention provides a computer program for determining the genotype of each gene locus of the particular gene loci or individual gene locus of a subject, which is characterized by including algorithms realizing the following functions (α) to (δ) to be executed in a computer.
(α) A function α, in which at least allele frequencies and depth of coverage of total reads of the gene loci or individual gene locus of the subject that were obtained by executing the computer program described before are retrieved.
(β) A function β, in which based on the allele frequencies of the gene loci or the individual gene locus retrieved by executing function α, calculation of the individual depth of coverage of each allele of the gene loci or individual gene locus, and allocation of the calculated individual depth of coverage to the alleles of the gene loci or the individual gene locus, are executed.
(γ) A function γ, in which given a rejection threshold value, which is set as a frequency number of 5 to 50%, preferably 10 to 30% of the average depth of coverage of all reads, the process in which alleles of the gene loci or individual gene locus whose individual depth of coverage specified by the function β described before is smaller than the threshold value are excluded from candidates for genotyping.
(δ): A function δ specified as (δ)-1 and (δ)-2 below.
(δ)-1 After the exclusion process in function (γ), for the case that there is one allele that was selected for genotyping the gene locus of the particular gene loci or individual gene locus, if the individual depth of coverage of the allele is twice or more of the rejection threshold value described before, the process in which it is determined that the genotype is homozygous of the allele, and if it is less than the twice of the rejection threshold, it is determined that the genotype is heterozygous of the allele, is executed, and
(δ)-2 After the exclusion process in function (γ), for the case that there are two alleles that were selected for genotyping the gene locus of the particular gene loci or individual gene locus, if the individual depth of coverage of the one whose individual depth of coverage is larger is less than twice of the smaller one, the process in which it is determined that the genotype is heterozygous of the two alleles, or if the individual depth of coverage of the one whose individual depth of coverage is larger is twice or more of the smaller one, it is determined that the genotype is homozygous of the allele whose individual depth of coverage is larger, is executed.

In addition to the processes described above, for example, it is also possible to add a function explicitly, in which for the case that there is no allele found for the genotype of each locus of the particular gene loci or individual locus after execution of exclusion process in the function (γ), the genotype of the gene locus based on the allele is not determined, is executed.

The computer program of the present invention can be described in, for example, C language, Java (registered trademark), Perl, Python, and other languages.

The present invention further provides a recording medium readable by a computer or a recording medium that can be connected to a computer (hereinafter also referred to as a recording medium of the present invention), which is characterized by that the program of the present invention is recorded. As the recording media, magnetic media such as flexible disks, flash memories, and hard disks, optical media such as CDs, DVDs, and BDs, magneto-optical media such as MO and MD, and other media can be mentioned, but the recording media of the present invention is not particularly limited to the examples. A typical computer system of the present invention is characterized by executing the program of the present invention.

### Effect of the Invention

The present invention provides the method to optimize the read mapping information of the particular gene loci or individual gene locus derived from read information obtained with a high-throughput sequencer for determining genotype of the gene loci or individual gene locus, the computer system for the optimization, the computer program for the optimization, the determination method for genotyping each gene locus of the gene loci and individual gene locus by applying the optimization method, the computer system for the determination, and the computer program for the determination. The present invention enables genotyping of a gene locus of gene loci or individual gene locus that are known to have similar nucleotide sequence in a genome or known to be highly polymorphic, in a high accurate and efficient manner. As the gene loci, for example, HLA gene loci, HAL being human MHC, cytochrome P450 loci, immunoglobulin gene loci, T cell receptor gene loci, and olfactory receptor gene loci can be considered.

### BRIEF DESCRIPTION OF THE DRAWINGS

FIG. 1 is a diagram showing a process flow of the present invention.

FIG. 2-1 is a flow sheet showing one embodiment of the optimization process of HLA-read mapping information with a latent variable Zₙₜ.

FIG. 2-2 is a flow sheet of the EM algorithm showing one embodiment of the optimization process of HLA-read mapping information with a latent variable Zₙₜₛ.

FIG. 2-3 is a flow sheet of the variational Bayesian algorithm showing one embodiment of the optimization process of HLA-read mapping information with a latent variable Zₙₜₛ.

FIG. 3 is a flow sheet showing one example of the HLA typing process based on “the fraction of reads allocated to each HLA allele” obtained through the above optimization process.

FIG. 4 is a figure showing prediction accuracy of HLA types at four-digit resolution at various depths of coverage using the simulation data.

FIG. 5-1 is a graph showing the results of examining allele frequencies of HLA-A by the system of the present invention and another method.

FIG. 5-2 is a graph showing the results of examining allele frequencies of HLA-B by the system of the present invention and another method.

FIG. 5-3 is a graph showing the results of examining allele frequencies of HLA-C by the system of the present invention and another method.

### MODES OF CARRYING OUT THE INVENTION

[A] Estimation Methods with the EM Algorithm and Variational Bayesian Method

Here, the contents of the EM algorithm and the variational Bayesian method disclosed in the previous application will be described in more detail. Symbols to be used are as described before unless otherwise noted.

### (1) EM (Expectation-Maximization) Algorithm

The EM algorithm is a method for obtaining the maximum likelihood estimate of the parameter in the probability model in which the latent variable exists. In the estimation method of the present invention, for example, it can be done with the following contents.

That is, the estimation method of the present invention by the EM method is exemplified as the estimation method of the present invention characterized by performing the following steps (a) to (e) (this method is also referred to as “the EM method of the present invention”).
(a) A step in which the first updated value of a posterior probability of Zₙₜₛ=1 or Zₙₜ=1 is calculated, based on a given initial value of θₜ, and the first updated value of the maximum likelihood estimate of θₜ is further calculated, based on the first updated value of the posterior probability of Zₙₜₛ=1 or Zₙₜ=1, or, (b) A step in which an updated value of the maximum likelihood estimate of θₜ is calculated, based on a given initial value of the posterior probability of the Zₙₜₛ=1 or Zₙₜ=1,
(c) A step in which an updated value of the posterior probability of Zₙₜₛ=1 or Zₙₜ=1 is calculated, based on the updated value of the maximum likelihood estimate of θₜ calculated by the previous step (d) (however, for the first time, it is step (a) or step (b)),
(d) A step in which an updated value of the maximum likelihood estimate of θₜ is calculated, based on the updated value of the posterior probability of Zₙₜₛ=1 or Zₙₜ=1 calculated in step (c), and
(e) (i) A step in which convergence of the log likelihood is evaluated by calculating the log likelihood based on the posterior probability of Zₙₜₛ=1 or Zₙₜ=1 calculated in step (c) and θₜ calculated in step (d),

(ii) A step in which convergence of the updated posterior probability of Zₙₜₛ=1 or Zₙₜ=1 calculated in step (c) is evaluated, or

(iii) A step in which convergence of the updated maximum likelihood estimate of θₜ calculated in step (d) is evaluated, and,

if convergence is recognized, θₜ in each step is determined as the final estimate value, and if convergence is not recognized, iteration of steps (c), (d) and (e) is determined.

The step (a) described above is a step of defining the initial value of this method for iterative optimization.

The initial value of n in Zₙₜₛ=1 or Zₙₜ=1 is a number from 1 to N, t is a number from 1 to T. The value of s is a number 1≦s≦lₜ−L+1, where lₜ is the length of allele t of the particular gene locus, L is the read length. If a read is mapped to multiple locations, it is preferable to set the initial value of Zₙₜₛ=1 or Zₙₜ=1 as 1/[The number of locations to which read n is mapped] by assuming that the mappings are equally likely. It is preferable to set the initial value of θₜ as 1/T by assuming that an allele frequency of each gene locus is the same. When the prior information of allele frequency of a specific gene locus is already known, it is also possible to use this information as the initial value of θₜ, for example, when the subject population is a specific racial group.

The step (b) is a step in which rₙₜₛ, the posterior probability of Zₙₜₛ=1 or rₙₜ, the posterior probability of Zₙₜ=1, is calculated based on the current estimated value (for the first iteration, it is calculated based on the initial value obtained in the step (a)).

That is, it is a step of calculating P (Zₙₜₛ=1|θ*ₜ) or P (Zₙₜ=1|θ*ₜ) (where * indicates that θₜ has been updated. The same notation follows below.), in which rₙₜₛ is calculated as

$$\begin{matrix}[ Mathematical 13 ] & \\ {r}_{nts} = \{ \begin{matrix}\frac{{ρ}_{nts}}{{\sum }_{( {t}^{′} , {s}^{′} ) ∈ {π}_{n}} {ρ}_{{nt}^{′} {s}^{′}}} & if ( t , s ) ∈ {π}_{n} \\ 0 & otherwise\end{matrix} . & \end{matrix}$$

Here, for the single-end reads, ρₙₜₛ is calculated as

log ρₙₜₛ=log θₜ+log p(Sₙ|Tₙ)+log p(Rₙ|Tₙ,Sₙ) [Mathematical 14]

(where the first term on the right side can be calculated by taking the logarithm of Expression (1) (a), the second term can be calculated by taking the logarithm of Expression (1) (c), and the third term can be calculated by taking the logarithm of Expression (2).)

For the paired-end reads, ρₙₜₛ is calculated as

$$\begin{matrix} [ Mathematical 15 ] & \\ log {ρ}_{nts} = {\sum }_{f = L}^{{l}_{t} - s + 1} \{ log {θ}_{t} + log p ( {F}_{n} = f \vert {T}_{n} ) + log p ( {S}_{n} \vert {T}_{n} , {F}_{n} = f ) + log p ( {R}_{na} \vert {T}_{n} , {S}_{n} ) + log p ( {R}_{nb} \vert {T}_{n} , {F}_{n} , {S}_{n} ) \} & \end{matrix}$$

(where the first term within the brackets on the right side can be calculated by taking the logarithm of Expression (3) (a), the second term can be calculated by taking the logarithm of Expression (3) (a′), the third term can be calculated by taking the logarithm of Expression (3) (b), the fourth term can be calculated by taking the logarithm of Expression (4-1), and the fifth term can be calculated by taking the logarithm of Expression (4-2).)

On the other hand, because Zₙₜ considers all the Zₙₜₛ described above with respect to all the possible s for each t, rₙₜ can be calculated as

$$\begin{matrix}[ Mathematical 16 ] & \\ {r}_{nt} = {\sum }_{s} {r}_{nts} & \end{matrix}$$

This corresponds to the E step of the EM method of the present invention.

The step (c) is a step corresponding to the M step of EM method of the present invention, in which based on rₙₜₛ or rₙₜ which is the posterior probability of Zₙₜₛ=1 or Zₙₜ=1 obtained in step (b), the maximum likelihood estimate θₜ that maximizes the log likelihood is calculated as follows.

$$\begin{matrix}[ Mathematical 17 ] & \\ {θ}_{t} = \frac{{\sum }_{{n}^{′} , {t}^{′} = t , {s}^{′}} {r}_{{n}^{′} {t}^{′} {s}^{′}}}{N} \\ or , & \\ [ Mathematical 18 ] & \\ {θ}_{t} = \frac{{\sum }_{{n}^{′} , {t}^{′} = t} {r}_{{n}^{′} {t}^{′}}}{N} & \end{matrix}$$

The steps (d) and (e) are as described above. As a preferred example of the convergence criterion, the relative change 10⁻³ is used as the convergence criterion for allele frequencies of the particular gene loci or individual gene locus for the abundance parameter θₜ>10⁻⁷, but different convergence criteria can be also used.

Usually, “iteration of steps (c), (d), and (e)” in step (e) is performed once or more, but it is also possible to do genotyping of alleles of the particular gene loci or individual gene locus without executing the iterative step, or with terminating the step when parameters are not converged.

### (2) Variational Bayesian Method

Variational Bayesian method is a method for estimating the posterior probability distribution of parameters by Bayesian estimation method, which enables robust estimation of parameters against noise.

The mathematical expression of the complete likelihood and posterior joint distribution of the estimation method of the present invention represented by the expression (1) involves integration over all possible latent variables Z and can not be solved analytically. Therefore, in the complete likelihood and posterior joint distribution, to obtain approximate solutions, the factorization of the latent variables and the model parameters is assumed as below.

q(θ, Z)≈q(θ)·q(Z) [Mathematical 19]

In the present invention, we use Dirichlet distribution as a prior distribution of θ, as described above.

$$\begin{matrix}[ Mathematical 20 ] & \\ p ( θ ) = \frac{1}{G ( α )} {\prod }_{t = 1}^{T} {θ}_{t}^{{α}_{t} - 1} & {( III )}^{′}\end{matrix}$$

[where Π^{T}ₜ₌₁θₜ=1, T is the number of alleles of the particular gene loci, αₜ is a hyperparameter,

$$\begin{matrix}[ Mathematical 21 ] & \\ G ( α ) = \frac{{\prod }_{t} Γ ( {α}_{t} )}{Γ ( {\sum }_{t} {α}_{t} )} , & \end{matrix}$$

and Γ(·) is gamma function.]

In the present invention, based on the assumption that there is no prior knowledge on the relative differences in the allele frequencies of particular gene loci or an individual gene locus such as HLA loci or locus, HLA being human a MHC, it is preferred that a single hyperparameter α₀ is used for all the alleles of the particular gene loci or individual gene locus. The single hyperparameter α₀ controls the complexity of the target parameter, that is, the number of θₜ>0. When α₀≧1, α₀−1 can be interpreted as a prior count of the read assigned to the allele of the particular gene loci or individual gene locus, and when α₀<1, the prior distribution favors a tendency for some of the allele frequencies at the particular gene loci or individual gene locus to be zero. Specifically, it is preferable to set 0<α₀≦0.1, or α₀ that maximizes the lower bound of the log marginal likelihood. When prior information of allele frequencies is known, it is also possible to weight αₜ>0 so that αₜ whose allele frequency is higher in prior information is given a larger value in descending order for each t. Given the hyperparameter α₀, the lower bound of the log marginal likelihood is iteratively maximized by the variational Bayesian estimation algorithm.

The estimation method of the present invention by variational Bayesian method is exemplified as the estimation method of the present invention characterized by carrying out the following steps (a) to (d) (this method is referred to as a “variational Bayesian method of the present invention”).
(a) A step in which the first update posterior distribution of Zₙₜₛ or Zₙₜ is calculated, based on a given initial posterior distribution of θₜ based on a given initial value of the hyperparameter α₀ representing prior information of allele frequencies of the particular gene loci or individual gene locus, and the first updated posterior distribution of θₜ is further calculated, based on the first updated posterior distribution of Zₙₜₛ or Zₙₜ, or, (b) A step in which the first updated posterior distribution of θₜ is calculated, based on a given initial distribution of Zₙₜₛ or Zₙₜ,
(c) A step in which an updated posterior distribution of Zₙₜₛ or Zₙₜ is calculated, based on the updated posterior distribution of θₜ calculated by the previous step (d) (however, for the first time, it is step (a) or step (b)),
(d) A step in which an updated posterior distribution of θₜ is calculated, based on the updated posterior distribution of Zₙₜₛ or Zₙₜ calculated in step (c), and
(e) A step in which convergence of an expected value of the updated posterior distribution of θₜ calculated in step (d) is evaluated, and if convergence is recognized, the expected value of θₜ is determined as the final estimate value, and if convergence is not recognized, iteration of steps (c), (d) and (e) is determined.

The step (a) described above is a step of defining the initial value of this method for iterative optimization. Specifically, it is preferable to set α*ₜ=(N/T+α₀)/Σₜ(N/T+α₀) for each allele t of the particular gene loci or individual gene locus, where α*ₜ is a parameter of q*(θ), assuming that each allele frequency of the particular gene loci or individual gene locus is equal.

The step (b) is a step to calculate E_{z} [Zₙₜₛ] or E_{z} [Zₙₜ] given the current estimate of q*(θ). The value for n in E_{z} [Zₙₜₛ] is the number from 1 to N, the value for t is the number from 1 to T, the value for s is the number 1≦s≦lₜ−L+1, where lₜ is the length of allele t of the particular gene loci or individual gene locus, and L is the read length. E_{z}[Zₙₜₛ] is calculated based on the current estimate of q*(θ) as below.

$$\begin{matrix}[ Mathematical 22 ] & \\ {E}_{Z} ( {Z}_{nts} ] = \{ \begin{matrix}\frac{{ρ}_{nts}}{{\sum }_{( {t}^{′} , {s}^{′} ) ∈ {π}_{n}} {ρ}_{{nt}^{′} {s}^{′}}} & if ( t , s ) ∈ {π}_{n} \\ 0 & otherwise\end{matrix} & \end{matrix}$$

Here, for the single-end reads, ρₙₜₛ is calculated as

log ρₙₜₛ=E_{θ}[log θₜ]+log p(Sₙ|Tₙ)+log p(Rₙ|Tₙ,Sₙ) [Mathematical 23]

(where the first term on the right side can be calculated by taking an expected value of the logarithm of Expression (1)(a), the second term can be calculated by taking the logarithm of Expression (1)(c), and the third term can be calculated by taking the logarithm of Expression (2).)

For the paired-end reads, ρₙₜₛ is calculated as

$$\begin{matrix} [ Mathematical 24 ] & \\ log {ρ}_{nts} = {\sum }_{f = L}^{{l}_{t} - s + 1} \{ {E}_{θ} [ log {θ}_{t} ] + log p ( {F}_{n} = f \vert {T}_{n} ) + log p ( {S}_{n} \vert {T}_{n} , {F}_{n} = f ) + log p ( {R}_{na} \vert {T}_{n} , {S}_{n} ) + log p ( {R}_{nb} \vert {T}_{n} , {F}_{n} , {S}_{n} ) \} & \end{matrix}$$

(where the first term within the brackets on the right side can be calculated by taking an expected value of the logarithm of Expression (3)(a), the second term can be calculated by taking the logarithm of Expression (3)(a′), the third term can be calculated by taking the logarithm of Expression (3)(b), the fourth term can be calculated by taking the logarithm of Expression (4-1), and the fifth term can be calculated by taking the logarithm of Expression (4-2).)

On the other hand, because Zₙₜ considers all the Zₙₜₛ described above with respect to all the possible s for each t, rₙₜ can be calculated as

$$\begin{matrix}[ Mathematical 25 ] & \\ {r}_{nt} = {\sum }_{s} {r}_{nts} & \end{matrix}$$

It is also possible to perform a series of calculations using Zₙₜ instead of Zₙₜₛ, and to execute the subsequent steps.

Here,

$$\begin{matrix}[ Mathematical 26 ] & \\ {E}_{θ} [ log {θ}_{t} ] = ψ ( {α}_{t}^{*} ) - ψ ( {\sum }_{t} {α}_{t}^{*} ) & \end{matrix}$$

(where ψ(·) is digamma function.)

The step (c) is a step of calculating E_{θ}[θₜ] given the current estimate of q*(Z).

E_{θ}[θₜ] is calculated based on the current estimate of q*(Z) as follows.

$$\begin{matrix}[ Mathematical 27 ] & \\ {E}_{θ} [ {θ}_{t} ] = \frac{{α}_{t}^{*}}{{\sum }_{{t}^{′}} {α}_{{t}^{′}}^{*}} \\ ( where {α}_{t}^{*} = {α}_{0} + {\sum }_{{n}^{′} , {t}^{′} = t , {s}^{′}} {E}_{Z} [ {Z}_{{n}^{′} , {t}^{′} , {s}^{′}} ] ) & \end{matrix}$$

Therefore, q*(θ) is also a Dirichlet distribution, and the prior distribution p(θ) is a conjugate prior distribution.

In the steps (d), as a preferred example of the convergence criterion, the relative change 10⁻³ is used as the convergence criterion for allele frequencies of the particular gene loci or individual gene locus for the expected value of the abundance parameter, E_{θ}[θₜ]>10⁻⁷, but different convergence criteria can be also used.

Here, we investigate the lower bound of the log marginal likelihood and show that the converged value obtained by updating the variational Bayesian method of the present invention approximates the true objective parameter θ.

The log marginal likelihood of the present invention is decomposed as

$$\begin{matrix}[ Mathematical 28 ] & \\ log p ( R ) = L ( q ) + KL ( q ‖ p ) \\ ( where & \\ [ Mathematical 29 ] & \\ L ( q ) = \int \int q ( θ , Z ) log \frac{p ( R , θ , Z )}{q ( θ , Z )} d θ dZ , \\ KL ( q ‖ p ) = - \int \int q ( θ , Z ) log \frac{p ( θ , Z \vert R )}{q ( θ , Z )} d θ dZ ) & \end{matrix}$$

Since KL (q∥p) is the Kullback-Leibler divergence between q(θ, Z) and p(θ, Z|R), the lower bound of the log marginal likelihood is given by L(q). That is, since the Kullback-Leibler divergence is always 0 or more, L(q) constitutes the lower bound of the log marginal likelihood. It is generally proved that L(q) (the lower bound of the log marginal likelihood) is increased each time the steps (b) and (c) are repeatedly updated (Bishop C M. Pattern Recognition and Machine Learning. Springer Science: Business Media, LLC, New York, N.Y., USA; 2006).

Based on the factorization assumption

q(θ, Z)≈q(θ) q(Z) [Mathematical 30]

as described above, L(q) is calculated as follows.

$$\begin{matrix}[ Mathematical 31 ] & \\ \begin{matrix}L ( q ) = E [ log p ( R , θ , Z ) ] - E [ log p ( θ , Z ) ] \\ = E [ log p ( R , Z \vert θ ) ] + E [ log p ( θ ) ] - E [ log q ( θ ) ] - E [ log q ( Z ) ]\end{matrix} \\ ( where & \\ [ Mathematical 32 ] & \\ E [ log p ( R , Z \vert θ ) ] = {\sum }_{n , t , s} {E}_{Z} [ {Z}_{nts} ] log {ρ}_{nts} , \\ E [ log p ( θ ) ] = {\sum }_{t} ( {α}_{0} - 1 ) {E}_{θ} [ log {θ}_{t} ] - log G ( α ) , \\ E [ log q ( θ ) ] = {\sum }_{t} ( {α}_{t}^{*} - 1 ) {E}_{θ} [ log {θ}_{t} ] - log G ( {α}^{*} ) , \\ E [ log q ( Z ) ] = {\sum }_{n , t , s} {E}_{Z} [ {Z}_{nts} ] log {E}_{Z} [ {Z}_{nts} ] ) & \end{matrix}$$

Usually, “iteration of steps (c), (d), and (e)” in step (e) is performed once or more, but it is also possible to do genotyping of alleles of the particular gene loci or individual gene locus without executing the iterative step, or with terminating the step when parameters are not converged.

(3) The Computer System and Computer Program using the EM Algorithm
(i) As a computer system using the EM algorithm described above, for example, the following computer system can be described. An embodiment of the computer system of the present invention is a computer system characterized in that a computer executes an estimation method using the EM algorithm described above.

The computer system of the present invention is a computer system for optimizing, for each read, the number of expected mappings to each allele of the gene loci or individual gene locus for the data where read mapping information of the particular gene loci or individual gene locus is mixed, comprising a recording unit and an arithmetic processing unit, which is characterized in that all or a part of the following processes (A) to (E), is executed:
(A) In the recording unit, read information of DNA derived from the subject is recorded as observed data of nucleotide sequence of read and alleles of the gene loci or individual gene locus to which the read is mapped to,
(B) In the arithmetic processing unit, based on the observed data retrieved from the recording unit, either one of the following initialization processes (B)-1 and (B)-2 is executed:

(B)-1: The process of calculating the initial value of θ, which represents allele frequency of the gene loci or individual gene locus, and

(B)-2: The process of calculating the initial values of two latent variables, (a) and (b) described below, that are related to the θ described above and the data of DNA read information of the subject, which is the observed data:

(a) A variable Tₙ that is dependent on θ, which represents allele selection of read n in the gene loci or individual gene locus, and

(b) The posterior probability of an indicator variable Zₙₜₛ (Zₙₜₛ equals to one if (Tₙ, Sₙ)=(t, s), and zero otherwise), which summarizes Sₙ, the start position of read n that is dependent on Tₙ, or Zₙₜ (Zₙₜ equals to one if Tₙ=t, and zero otherwise), which summarizes the latent variable Tₙ,
(C) In the arithmetic processing unit, based on the parameter θ calculated in the process (B)-1 described above, the calculation process of the posterior probability of indicator variable Zₙₜₛ or Zₙₜ=1 is exerted,
(D) In the arithmetic processing unit, the first updated value of the maximum likelihood estimate of parameter θ is calculated based on the posterior probability of indicator variable Zₙₜₛ or Zₙₜ=1 calculated in the process (B)-2 or (C),
(E) In the arithmetic processing unit, the processes (C) and (D) described above are executed again based on the first updated value of the maximum likelihood estimate of θ calculated in the process (D) described above, and the iterative loop process for calculating the second updated value of θ is repeatedly executed, until difference between the new updated value of θ and the previous updated value of θ is not recognized, and the converged parameter θ is recognized as the optimized value and recorded in the recording unit described above.
(ii) As a computer program using the EM algorithm described above, for example, the following computer program can be considered. The embodiment of the computer program of the present invention is a computer program characterized by realizing the estimation method using the EM algorithm in a computer.

The computer program is a computer program for optimizing, for each read, the number of expected mappings to each allele of the gene loci or locus for the data where read mapping information of the particular gene loci or individual locus is mixed, which is characterized in that all or a part of the following functions 1 to 5, is realized:
(A) Function 1 in which read information of DNA derived from the subject recorded as observed data of nucleotide sequence of read and alleles of the loci or locus to which the read is mapped to, is retrieved from the recording unit,
(B) Function 2 in which based on the observed data retrieved by function 1 described above, either one of the following initialization processes (B)-1 and (B)-2 is executed:

(B)-1: A process of calculating the initial value of θ, which represents allele frequency of the gene loci or individual gene locus, and

(B)-2: A process of calculating the initial values of two latent variables, (a) and (b) described below, that are related to the θ described above and the data of DNA read information of the subject, which is observed data:

(a) A variable Tₙ that is dependent on θ, which represents allele selection of read n in the gene loci or individual gene locus, and

(b) The posterior probability of an indicator variable Zₙₜₛ (Zₙₜₛ equals to one if (Tₙ, Sₙ)=(t, s), and zero otherwise), which summarizes Sₙ, the start position of read n that is dependent on Tₙ, or Zₙₜ (Zₙₜ equals to one if Tₙ=t, and zero otherwise), which summarizes the latent variable Tₙ,
(C) Function 3 in which based on the parameter θ calculated in the process (B)-1 described above, the calculation process of the posterior probability of indicator variable Zₙₜₛ or Zₙₜ is executed,
(D) Function 4 in which the first updated value of the maximum likelihood estimate of parameter θ is calculated based on the posterior probability of indicator variable Zₙₜₛ=1 or Zₙₜ=1 calculated in the process (B)-2 or function 3,
(E) Function 5 in which function 3 and 4 described above are executed again based on the first updated value of the maximum likelihood estimate of θ calculated in the function 4 described above, and the iterative loop process for calculating the second updated value of θ is repeatedly executed, until difference between the new updated value of θ and the previous updated value of θ is not recognized, and the converged parameter θ is recognized as the optimized value and recorded in the recording unit described above.
   (4) The Computer System and Computer Program using the Variational Bayesian Method
(i) As a computer system using the variational Bayesian method described above, for example, the following computer system can be considered. The embodiment of the computer system of the present invention is a computer system characterized by realizing the estimation method using the variational Bayesian method in a computer.

The computer system of the present invention is a computer system for optimizing, for each read, the number of expected mappings to each allele of the gene loci or locus for the data where read mapping information of the particular gene loci or individual gene locus is mixed, comprising a recording unit and an arithmetic processing unit, which is characterized in that all or a part of the following processes (A) to (E), is executed:
(A) In the recording unit, read information of DNA derived from the subject is recorded as observed data of nucleotide sequence of read and alleles of the loci or locus to which the read is mapped to,
(B) In the arithmetic processing unit, based on the observed data retrieved from the recording unit, either one of the following initialization processes (B)-1 and (B)-2 is executed:

(B)-1: A process of calculating the updated value of the posterior distribution of θₜ, based on the initial value of hyperparameter αₜ which represents prior information of allele frequency of the gene loci or individual gene locus, and

(B)-2: A process of calculating the initial distribution of two latent variables, (a) and (b) described below, that are related to the distribution of θ described above and the data where DNA read information of the subject is mixed, which is the observed data:

(a) A variable Tₙ that is dependent on θ, which represents allele selection of read n in the gene loci or individual gene locus,

(b) The posterior distribution of an indicator variable Zₙₜₛ (Zₙₜₛ equals to one if (Tₙ, Sₙ)=(t, s), and zero otherwise), which summarizes Sₙ, the start position of read n that is dependent on Tₙ, or Zₙₜ (Zₙₜ equals to one if Tₙ=t, and zero otherwise), which summarizes the latent variable Tₙ,
(C) In the arithmetic processing unit, based on the distribution of parameter θ calculated in the process (B)-1 described above, the calculation process of the posterior distribution of indicator variable Zₙₜₛ or Zₙₜ is exerted,
(D) In the arithmetic processing unit, the first updated posterior distribution of parameter θ is calculated based on the updated posterior distribution of Zₙₜₛ or Zₙₜ calculated in the process (B)-2 or (C),
(E) In the arithmetic processing unit, the processes (C) and (D) described above are executed again based on the first updated posterior distribution of θ calculated in the process (D), and the iterative loop process in which the second updated posterior distribution of θ is calculated is repeatedly executed, until difference between the expected value of the new updated posterior distribution of θ and the expected value of the previous updated posterior distribution of θ is not recognized, and the converged expected value of the posterior distribution of θ is recognized as the optimized value and recorded in the recording unit described above.
(ii) As a computer program using the variational Bayesian method described above, for example, the following computer program can be considered. The embodiment of the computer program of the present invention is a computer program characterized by realizing the estimation method using the variational Bayesian method in a computer.

The computer program is a computer program for optimizing, for each read, the number of expected mappings to each allele of particular gene loci or individual gene locus for the data where read mapping information of the particular gene loci or individual gene locus is mixed, which is characterized in that all or a part of the following functions 1 to 5, is realized:
(A) Function 1 in which read information of DNA derived from the subject recorded as observed data of nucleotide sequence of read and alleles of the loci or locus to which the read is mapped, is retrieved from the recording unit,
(B) Function 2 in which based on the observed data retrieved by function 1 described above, either one of the following initialization processes (B)-1 and (B)-2 is executed:

(B)-1: A process of calculating the updated posterior distribution of θₜ, based on the initial value of hyperparameter αₜ which represents the prior information of allele frequency of the gene loci or individual gene locus, and

(B)-2: A process of calculating the posterior distribution of two latent variables, (a) and (b) described below, that are related to the distribution of θ described above and the data where DNA read information of the subject is mixed, which is the observed data:

(a) A variable Tₙ that is dependent on θ, which represents allele selection of read n in the gene loci or individual gene locus,

(b) The initial posterior distribution of an indicator variable Zₙₜₛ (Zₙₜₛ equals to one if (Tₙ, Sₙ)=(t, s), and zero otherwise), which summarizes Sₙ, the start position of read n that is dependent on Tₙ, or Zₙₜ (Zₙₜ equals to one if Tₙ=t, and zero otherwise), which summarizes the latent variable Tₙ,
(C) Function 3 in which based on the distribution of θ calculated in the process (B)-1 described above, the calculation process of the posterior distribution of indicator variable Zₙₜₛ or Zₙₜ is executed,
(D) Function 4 in which the first updated value of the posterior distribution of θ is calculated based on the updated posterior distribution of Zₙₜₛ or Zₙₜ calculated in the process (B)-2 or function 3,
(E) Function 5 in which function 3 and 4 described above are executed again based on the first updated value of the posterior distribution of θ calculated in the function 4 described above, and the iterative loop process in which the second updated value of the posterior distribution of θ is calculated is repeatedly executed, until difference between the expected value of the new updated posterior distribution of θ and the expected value of the previous updated posterior distribution of θ is not recognized, and the converged expected value of θ is recognized as the optimized value and recorded in the recording unit described above.

### [B] A More Specific Form of the Method of the Present Invention

Here, we disclose an example where the particular gene loci are HLA loci, HLA being a human MHC, but the algorithm disclosed herein may be applied to other loci, cytochrome P450 loci, immunoglobulin gene loci, T cell receptor gene loci, and olfactory receptor gene loci. The method of the present invention is used as a generic name of the optimization method and determination method, as described above.

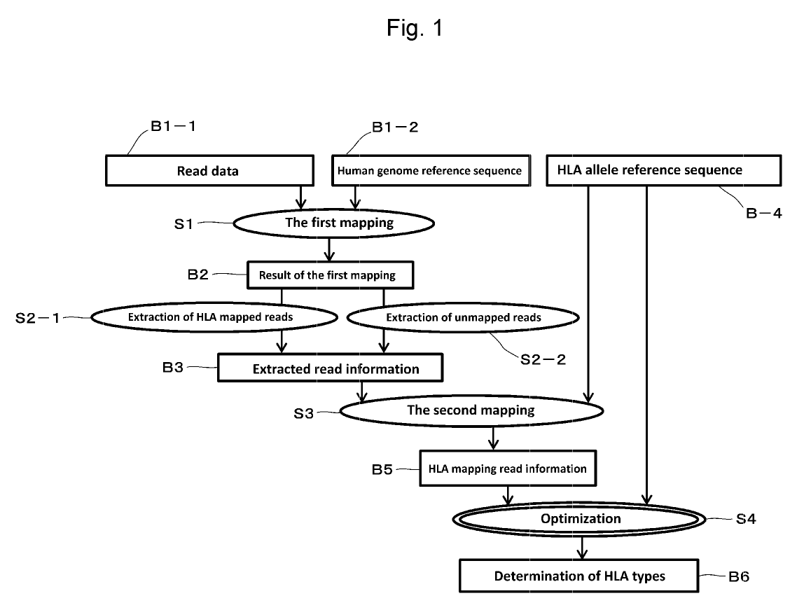

FIG. 1 is a process flow of the method of the present invention for predicting the genotype of a subject's HLA locus. Each element of this flow of processing is all performed electronically on the computer based on an instruction to realize an algorithm of computer software via a computer network or a database as necessary.

Box B1-1 indicates the presence of read data and box B1-2 indicates the presence of a human genome reference sequence. As described above, the “read data” (box B1-1) is electronic information of nucleotide sequence of a part or all of the individual DNA fragments (reads) obtained by processing the subject's DNA sample with a high-throughput sequencer, and normally, information indicating the reading accuracy is also added (FASTQ: a term derived from the FASTA format representing the nucleotide sequence of DNA). At this stage, “depth of coverage of total reads” is determined. The “human genome reference sequence” (box B1-2) is constructed from one or multiple human genome sequence information as described above, and its source of supply is not particularly limited here. It is also assumed that the ethnicity of the subject and that of individuals from which the human genome reference sequence is constructed are different, and there exists heterogeneity with respect to the genome sequence in HLA genes. The substantial effect of the present invention does not depend on the version change of the reference sequence, and it is always possible to determine HLA types of the subject in correspondence with the changes of the reference sequence, and as the latest reference sequence provided at present or the one that will be provided in the future can be used. Examples of human genome reference sequences include the human genome reference sequence (such as hg19) managed by UCSC (University of California Santa Cruz), the human genomic reference sequence (such as GRCh37) managed by the Genome Reference Consortium, and others, but it is not limited to these examples. As described above, for example, sequence data obtained from targeted sequencing, Exome sequencing, RNA sequencing, and long-read sequencing such as with PacBio RSII and Oxford Nanopore and others can also be used as a reference sequence.

The step S1 is a step of performing the first mapping, or, a step of mapping the “read data” of the box B1-1 to the “human genome reference sequence” of the box B1-2. With regard to this mapping, those skilled in this field can create computer programs including algorithms capable of realizing the mapping with computers, but it is also possible to use existing software. The existing mapping software includes, for example, BWA-MEM (http://bio-bwa.source.net/), Bowtie 2 (http://bowtie-bio.sourceforge.net/bowtie 2/index.shtml), Novoalign (http://www.novocraft.com/products/novoalign/), and others. Also, when the read data is paired-end sequence data, if one of the nucleotide sequences (pairs) of both ends of the read is mapped to the HLA locus and the other is not mapped, it is preferable to extract both reads of the pair and use them in the following processes.

Furthermore, it is preferable that a “decoy sequence” is included in addition to the human genome reference sequence described above. A “decoy sequence” is a genomic sequence that does not exist in a reference sequence prepared in advance. This is because, without using a decoy sequence, there is a high risk that reads that are not derived from sequences registered in the human genome reference sequence will be mapped to some places in the existing human genomic reference sequences, possibly reducing mapping accuracy. As a decoy sequence, hs37d5 (ftp: //ftp.1000genomes.ebi.ac.uk/vol1/ftp/technical/reference/phase2_reference_assembly_sequence/hs37d5.fa.gz) and others can be used.

The box B2 is a box showing the existence of “the result of the first mapping” in the above S1, and the mapping result exists in the format such as the SAM format or the BAM format.

Step S2-1 is a step of “extracting reads mapped to HLA loci” from “the result of the first mapping” in the box B2, and step S2-2 is a step of performing “extraction of unmapped reads” that are not mapped to any portion of the human genome reference sequence. With respect to the execution of these extraction, it is possible for those skilled in the field to create computer programs including algorithms that can realize the extraction with a computer, but it is also possible to use existing software. As existing extracting software, for example, SAMtools (http://samtools.sourceforge.net/) can be mentioned. In this extraction step, it is preferable to carry out calculation as efficient as possible because the necessary processing to be performed spans at least all HLA loci, which are many loci. Therefore, it is preferable that the computer program mentioned above is designed so that the extraction is carried out on all HLA loci (currently, HLA-A, -B, -C, -DM, -DO, -DP, -DQ, -DR, -E, -F, -G, -H, -J, -K, -L, -P, -V, -MIC, and -TAP are registered in the IMGT/HLA database). With SAMtools, extraction processing can be easily performed. It is preferable that “Extraction of unmapped reads” in step S2-2 is set as a step to prevent the loss of reads derived from HLA loci that were not mapped to the reference genomic sequence due to polymorphism in the subject's genomic sequence as described above.

Through these steps in S2, the information of reads that were mapped to regions other than HLA loci are excluded.

The box B3 is a box showing the presence of the read information (extracted reads) extracted in the above step S2 and the box B4 is a box showing the existence of the information of “reference sequence of HLA allele”. Reference sequence information of HLA allele is electronic information derived from a database in which genome information of HLA allele is stored. As the database, for example, IMGT/HLA (http://www.ebi.ac.uk/ipd/imgt/hla/), and others can be mentioned. It is preferable to acquire the latest update information from these database information as much as possible. In addition, it is preferable that HLA alleles of “pseudogenes” which are genes that do not actually code for proteins are also included in the genomic information of HLA allele.

Step S3 is a step in which “second mapping” to “reference sequence of HLA allele” in box B4 of “extracted read information” in box B3 is executed. It is preferable that the mapping software is designed so that the execution mode of the second mapping is performed so as to allow one read to be mapped to multiple HLA alleles, and it is possible by specifying “-a option” with BWA-MEM mentioned above and “-k option” with Bowtie 2, which make it possible for the computer to realize the multiple mapping of reads to HLA alleles.

Box B5 is a box showing “the result of the second mapping” in step S3, that is, the presence of “HLA mapping read information”, and the information exists in a format such as the SAM format or BAM format.

The “mapping process of the present invention” described above is shown until this step in FIG. 1.

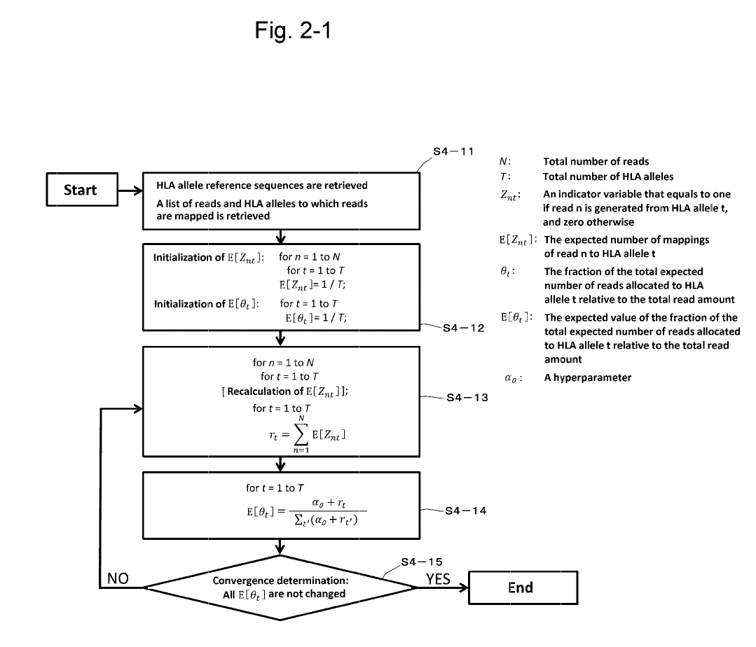

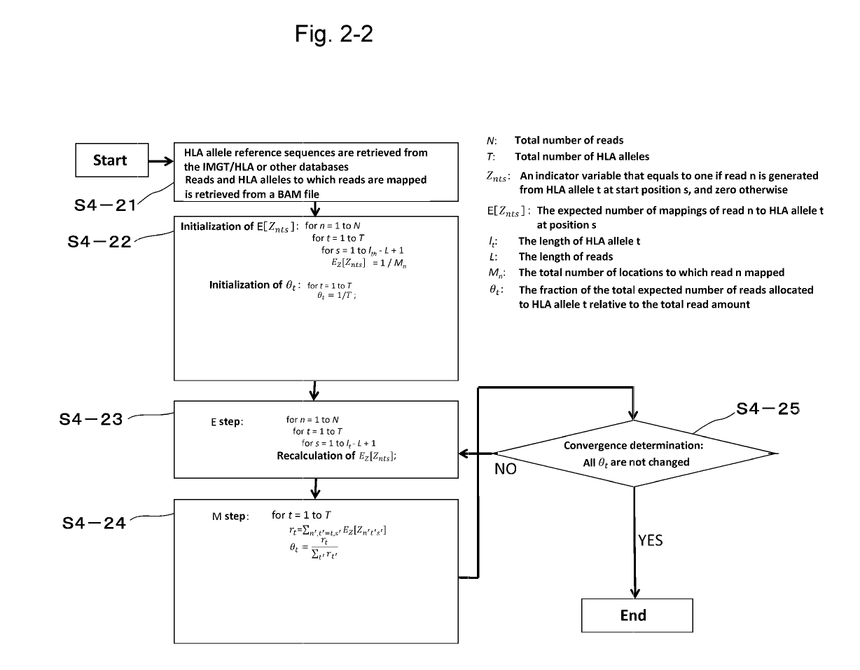

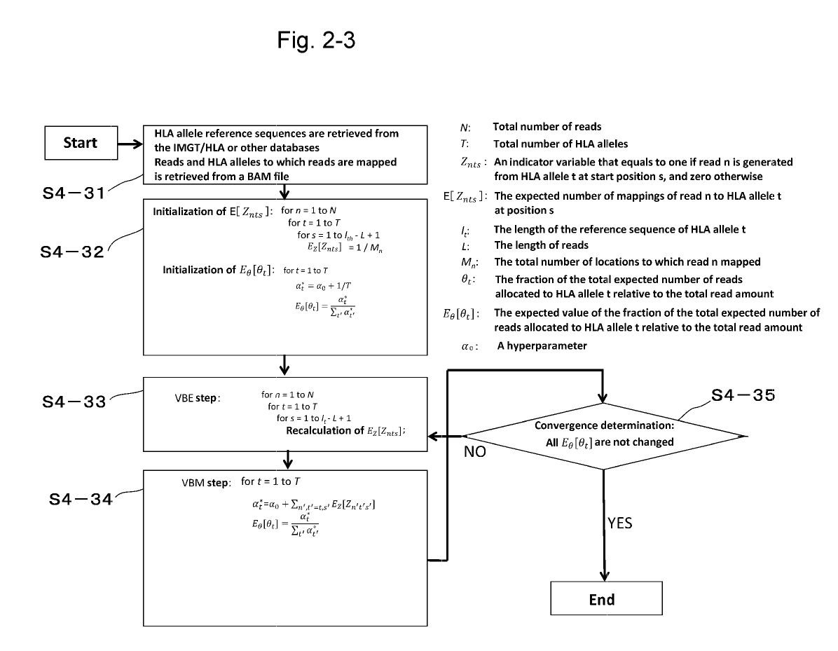

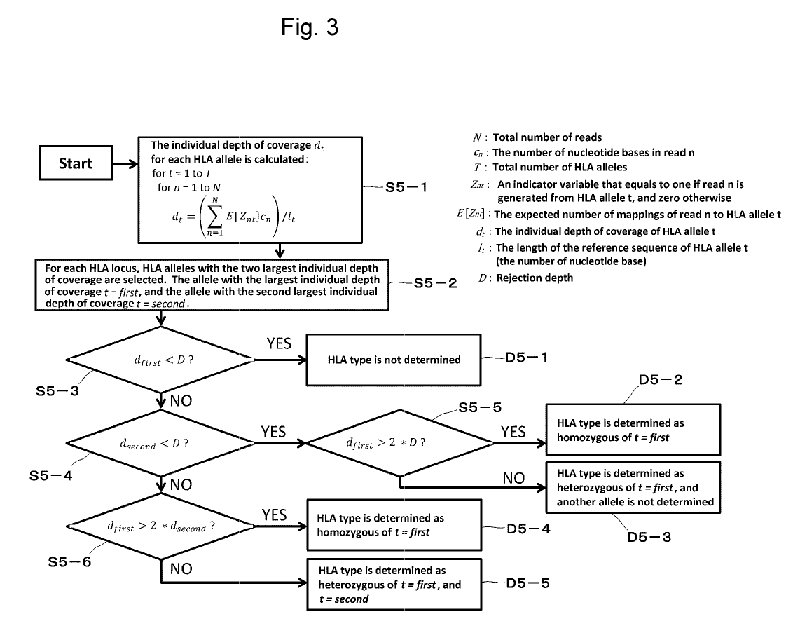

Hereinafter, the step S4 is a step of optimizing the “HLA mapping read information” described above, and details thereof are shown in FIG. 2 (FIGS. 2-1, 2-2, 2-3) as a flow sheet. FIG. 2-1 shows embodiments (variational Bayesian method and EM algorithm) of the optimizing method of the present invention using the latent variable Zₙₜ, FIG. 2-2 shows an embodiment using the EM algorithm in the optimization method of the present invention using the latent variable Zₙₜₛ, and FIG. 2-3 shows an embodiment using the variational Bayesian method in the optimization method of the present invention using the latent variable Zₙₜₛ. The detailed calculation steps involved in these embodiments are as described above for alleles of the particular gene loci. Furthermore, the box B6 is a box showing the presence of electronic information of the desired “Determination of HLA type”, but in the process from step S4 to box B6, the HLA typing process is preferably executed using the rejection threshold shown in FIG. 3 (described later).

[An Embodiment of the Optimization Method using the Latent Variable Zₙₜ]

FIG. 2-1 is a flow sheet showing one embodiment of the optimization process using the latent variable Zₙₜ of “HLA mapping read information” as described above. This flow sheet is an embodiment in which prediction by the variational Bayesian method is performed for Bayesian estimation to obtain expected read counts on HLA alleles, but it is also possible to perform prediction by the EM algorithm in order to perform maximum likelihood estimation, which does not need incorporation of the hyperparameter α₀ as described below.

Step S4-11 is a step of retrieving “HLA allele reference sequence” shown in the box B4 and retrieving “HLA mapping read information” shown in the box B5.

Step S4-12 is a step in which the number of expected mappings E[Zₙₜ] to each HLA allele of the read n used in the optimization process by executing the computer program of the present invention, and the expected value of the fraction of the total number of expected mappings allocated to each HLA allele relative to the total read amount, E[θₜ], are initialized.

Step S4-13 is a step in which the total number of expected mappings for each HLA allele for all reads based on the initialized values obtained in step S4-12 described above is calculated. The definition of the mathematical expression described here is as described above. The rₜ is “the total number of expected mappings assigned to HLA allele t”.

Step S4-14 is a step in which the “total number of expected mappings” for each HLA allele, calculated in step S4-13 described above is divided by the sum of the total number of expected mappings for all HLA alleles, and the expected value of the fraction of the total number of expected mappings allocated to each HLA allele relative to the total read amount, E[θₜ], is calculated. In this step S4-14, the hyperparameter α₀ has been incorporated as described above, in which an embodiment of the variational Bayesian method is described. For example, α₀=1 which gives a uniform distribution is used as the hyper parameter α₀ assuming that there is no prior knowledge, but α₀ that maximizes the lower bound of the log likelihood, for example, α₀=0.01 is also preferably used. Furthermore, as described above, it is also possible to use it as a flow sheet of an embodiment of the EM algorithm for performing maximum likelihood estimation of the parameter θₜ without incorporating this hyperparameter.

Step S4-15 is a step related to loop processing, and
(i) The function in which the distribution of the fraction of the total number of expected mappings calculated in step S4-14 described above is assigned to each HLA allele as a frequency distribution, and based on the allocated frequency distribution, again in step S4-13 described above, the number of expected mappings for each HLA allele for each read is calculated,
(ii) The function in which based on the number of expected mappings for each HLA allele for each read calculated in the function (i) described above, step S4-13 or S4-14 is executed again for calculating the updated distribution of the fraction of reads assigned to each HLA allele relative to the total read amount mapped to HLA alleles,
(iii) The function in which the calculation processes in the (i) and (ii) described above are repeatedly executed, until the difference between the number of expected mappings to each HLA allele for each read calculated by executing the current (i) and that calculated by executing the previous (i) is not recognized for all the reads, or, the difference between the expected value of the distribution of the fraction of the total number of expected mappings calculated by executing the current (ii) and that calculated by executing the previous (ii) is not recognized for all HLA alleles, and the converged expected number of mappings to each HLA allele for each read or the converged expected value of the distribution of the fraction of reads for each HLA allele is recognized as the optimized data as “convergence value”;
   are included in the description of step S4-15.
   [Embodiment of the Optimization Method using Latent Variable Zₙₜₛ (1)]

FIG. 2-2 is a flow sheet showing an embodiment of the optimization process using the latent variable Zₙₜₛ of “HLA mapping read information” as described above. This flow sheet describes prediction of the expected read counts on HLA alleles by maximum likelihood estimation with the EM algorithm.

Step S4-21 is a step of retrieving the “HLA mapping read information” in the same manner as in step S4-11 described above.

Step S4-22 is a step in which a latent variable Zₙₜₛ (a latent variable taking 1 in the case where the read n is generated from the position s of the HLA allele t) and θₜ, the fraction of reads allocated to each HLA allele relative to the total read amount, used in the optimization process by executing the computer program, are initialized. The expected value of the latent variable Zₙₜₛ is described as E[Zₙₜₛs]. Here, N is the “total number of reads”, and T is the “total number of HLA alleles”. lₜ is the length of the HLA allele t, and L is the read length. Mₙ is the total number of observed mapped locations of the read n. E[Zₙₜₛ] is initialized as 1/Mₙ, given an assumption of no prior information/uniform distribution. θₜ is given as 1/T as an initial value, given an assumption of no prior information/uniform distribution.

Step S4-23 is a step in which for all reads, the number of expected mappings to each HLA allele based on the initialized information retrieved in step S4-22 is calculated. That is, this is a step in which the expected value of the latent variable Zₙₜₛ (E_{z} [Zₙₜₛ]) indicating the allocation of read n to the position s of the HLA allele t, based on the θₜ updated immediately before (for the first time, it is the initial values in step S4-22 described above), that is, the “expected number of mappings”, is calculated, which corresponds to the E step of the EM method.

Step S4-24 is a step in which the “expected number of mappings” calculated in step S4-23 described above is summed for each HLA allele and the “total number of expected mappings” (rₜ=Σ_{n′,t′=t,s′} E_{z}[Z_{n′t′s′}]) is calculated, and the process in which the total number of expected mappings is divided by the sum of the total number of expected mappings for all HLA alleles to calculate the maximum likelihood estimate of the fraction of reads assigned to each allele relative to the total read amount “θₜ” (θₜ=rₜ/Σ_{t′}r_{t′}), is executed, which corresponds to the M step of the EM method. This step corresponds to calculating θₜ that locally maximizes the log likelihood.

Step S4-25 is a step related to loop processing. That is, steps S4-23 and S4-24 are executed again based on the first updated value of the maximum likelihood estimate of θₜ calculated in step S4-24, and the loop processing in which the second updated value of the parameter θ is calculated is repeatedly executed until the significant difference between the new updated value and the previously updated value is not recognized for all HLA alleles, and the converged parameter θ is recorded on the recording unit as the optimized θ.

[Embodiment of the Optimization Method using Latent Variable Zₙₜₛ (2)]

FIG. 2-3 is a flow sheet showing an embodiment of the optimization process using the latent variable Zₙₜₛ of “HLA mapping read information” as described above. This flow sheet describes prediction of the expected read counts on HLA alleles and HLA allele frequency by variational Bayesian method.

Step S4-31 is a step of retrieving the “HLA mapping read information” in the same manner as in step S4-11 described above.

Step S4-32 is a step in which the posterior distribution of a latent variable Zₙₜₛ (a latent variable taking 1 in the case where the read n is generated from the position s of the HLA allele t, and 0 otherwise) and the posterior distribution of θₜ (the fraction of reads allocated to each HLA allele relative to the total read amount), used in the optimization process by executing the computer program, are initialized. The expected value of the posterior distribution of the latent variable Zₙₜₛ is described as E[Zₙₜₛ]. Here, N is the “total number of reads”, and T is the “total number of HLA alleles”. lₜ is the length of the HLA allele t, and L is the read length. Mₙ is the total number of observed mapped locations of the read n. E[Zₙₜₛ] is initialized as 1/Mₙ, given an assumption of no prior information/uniform distribution. The expected value of θₜ, E_{θ}[θₜ], is given as α*ₜ/Σ_{t′}α*t′. Here, α*ₜ=α₀+N/T. As described above, α₀ is a hyperparameter. For example, assuming that there is no prior information, α₀=1 that gives a uniform distribution is used, but it is also preferable to use α₀, which maximizes the lower bound of log likelihood, for example, α₀=0.01.

Step S4-33 is a step in which for all reads, the number of expected mappings to each HLA allele based on the initialized information retrieved in step S4-32 is calculated. That is, this is a step in which the expected value of the posterior distribution of the latent variable Zₙₜₛ, E_{z}[Zₙₜₛ], indicating the allocation of read n to the position s of the HLA allele t, based on the posterior distribution of θₜ updated immediately before (for the first time, it is the initial values in step S4-32 described above), that is, the “expected number of mappings”, is calculated, which corresponds to the E step of the variational Bayesian method.

Step S4-34 is a step in which the posterior distribution of θₜ is calculated based on the “expected number of mappings” (E_{z}[Zₙₜₛ]) calculated in step S4-33 described above, which corresponds to the M step of the variational Bayesian method, and the expected value of the posterior distribution of θₜ, E_{θ}[θₜ], is calculated based on the E_{z}[Zₙₜₛ] calculated in step S4-34 as

$$\begin{matrix}[ Mathematical 33 ] & \\ {E}_{θ} [ {θ}_{t} ] = \frac{{α}_{t}^{*}}{{\sum }_{{t}^{′}} {α}_{{t}^{′}}^{*}} \\ ( where {α}_{t}^{*} = {α}_{0} + {\sum }_{{n}^{′} , {t}^{′} = t , {s}^{′}} {E}_{Z} [ {Z}_{{n}^{′} {t}^{′} {s}^{′}} ] ) & \end{matrix}$$

Step S4-35 is a step of selecting whether to repeat steps S4-33 and S4-34 described above, or to end the loop process.

That is, steps S4-33 and S4-34 are executed again based on the first updated posterior distribution of the parameter θₜ calculated in step S4-24, and the loop processing of calculating the second updated posterior distribution of the parameter θ is iteratively executed until the significant difference between the expected value of the newly updated posterior distribution and the expected value of the posterior distribution updated last time is not recognized, and the converged expected value of θ is recorded in the recording unit as the data of optimized θ.

It is possible to use the obtained “rₜ” or “θₜ” as an indicator of HLA typing, but it is preferable that the following HLA typing process is performed.

FIG. 3 is a flow sheet showing an example of the HLA typing process using a rejection threshold for the individual depth of coverage that is determined relative to the depth of coverage of all reads, based on “the fraction of reads assigned to each HLA allele t “θₜ”, or the expected value of the read fraction “θₜ””, obtained through the optimization step described above.

Step S5-1 is a step in which the “individual depth of coverage” of each HLA allele is calculated from “the fraction of reads assigned to each HLA allele”. The variables “t” and “n” are initialized, and the process of calculating the individual depth “dₜ” is performed using the mathematical expression indicated in box S5-1. The details of the calculation process here have already been described.

Step S5-2 is a step in which, as described in the box, for each HLA locus, two of the HLA alleles with the two largest individual depth of coverage “dₜ” are selected, and the allele with the largest individual depth of coverage, and the allele with the second largest individual depth of coverage are specified.

Step S5-3 is a step in which a selection process to determine whether the largest individual depth of coverage “d_{first}” of the allele described above is smaller than the rejection depth “D” or not is executed. The rejection depth “D” is a numerical value selected between 5 and 50%, preferably between 10 and 30% of the “depth of coverage of total reads” as described above. If the individual depth of coverage “d_{first}” is smaller than the rejection depth “D”, a decision is made that “HLA type is not determined” (conclusion D5-1). If it is not smaller, the next selection step S5-4 is executed.

Step S5-4 is a step in which a selection process to determine whether the second largest individual depth of coverage “d_{second}” of the allele described above is smaller than the rejection depth “D” or not is executed. If the individual depth of coverage “d_{second}” is smaller than the rejection depth “D”, a decision is made to go to the selection step S5-5, and if it is not smaller, a decision is made to go to the selection step S5-6.

Step S5-5 is a step that is applied when the individual depth of coverage “d_{second}” is smaller than the rejection depth “D”. That is, a determination process in which if the “d_{first}” described above is greater than twice the rejection depth “D”, “the HLA type is homozygous of the HLA allele with the largest individual depth of coverage” (conclusion D5-2), and if it is not larger than 2 times, it is assumed that “the HLA type is determined as heterozygous of the HLA allele with the largest individual depth of coverage and the other allele is not determined” (conclusion D5-3), is performed.

Step S5-6 is a further selection step that is applied when “d_{second}<D” is not satisfied in step S5-4, and a determination process in which if “d_{first}” is more than twice the individual depth of coverage “d_{second}”, “the HLA type is homozygous of the HLA allele with the highest individual depth of coverage” (conclusion D5-4), and if it is not more than twice, “the HLA type is heterozygous of the HLA alleles with the largest and the second largest individual depth of coverage” (conclusion D5-5), is performed.

As described above, the system of the present invention can determine the HLA type in the preferred form (box B6 in FIG. 1).

### EXAMPLES

Hereinafter, examples of the present invention performed in which the target is HLA, which is human MHC, are disclosed.

[A] The Source used in the Examples

The sources used in executing the computer system and the computer program (hereinafter collectively referred to as the “system of the present invention”) used in the present examples are as follows.

### (1) HLA Mapping Process of the Present Invention

HiSeq2000 (Illumina, Inc.) was used as the next-generation sequencer that provides the “read information” of box B1-1 in FIG. 1. The read information is provided in the FASTAQ format, and the subsequent read information is likewise in the FASTAQ format.

As the “human genome reference sequence” in box B1-2, hg19 (UCSC) or GRCh37 (Genome Reference Consortium) was used in conjunction with decoy sequence (hs37d5).

The mapping of step S1/S3 was performed by BWA-MEM. For step S3, the option “-a” was specified and all possible mapping locations were output for each read.

The “first mapping result” in box B2 and the “second mapping result” in box B5 were used in the BAM format.

As software for extracting the mapping result in step S2, “SAMtools” was used.

“Reference sequence of HLA allele” in box B4 was provided in the FASTA format obtained from the IMGT/HLA database.

### (2) The Optimization Process

The optimization process in step S4 is executed by applying the algorithm described in flow sheet in FIG. 2-1 using the variational Bayesian method to the paired-end data, the rejection depth is set to 20% of the depth of coverage of total reads, and the rejection process was executed by applying the algorithm described in the flow sheet in FIG. 3.

### [B] Performance Measurement of HLA Typing

### (1) Overview of the Simulation Experiment

The prediction performance of the system of the present invention was evaluated from the viewpoint of prediction accuracy. The prediction accuracy is defined as the fraction of the true positive prediction relative to the predicted HLA types. In this simulation experiment, two HLA alleles (either heterozygous or homozygous) for the six HLA loci (HLA-A, -B, -C, -DQA1, -DQB1, -DRB1) were evaluated in each individual. Predictive performance was evaluated separately for each method with two-digit, four-digit, six-digit and eight-digit resolution.

### (2) Simulation Data Analysis

With the simulation data analysis, the performance of the system of the present invention to predict HLA types was evaluated compared to other systems. As the comparable systems, (a) PHLAT (Bai et al., BMC genomics, 15: 325 (2014)) and (b) HLAminer (Warren et al., Genome medicine, 4 (12): 102 (2013)) were used. It is known that these comparable systems can classify HLA class I loci (HLA-A, -B, and -C) and the class II loci (HLA-DQA1, -DQB1, and -DRB1) at a four-digit resolution from human whole-genome sequence data.

First, simulation data of 1,000 individuals of human samples was prepared. For each individual, HLA diplotype of the six HLA loci was randomly selected from HLA alleles registered in the IMGT/HLA database release 3.15.0. Once the HLA type was established for each individual, one SNP for 1,000 bp was incorporated in each individual's HLA allele based on the average polymorphism in the human genome. Next, 100 bp of the paired-end read data (average depths of coverage were 5×, 10×, 20× and 30×, and the mean fragment length and standard deviation of the distribution were set as 300 bp and 40 bp, respectively) was uniformly produced across the HLA allele sequence with 0.1% substitution, deletion and insertion errors.

Table 1 shows the prediction accuracy of the system of the present invention and the existing tool using the 30× simulation data.

TABLE 1

| HLA | System of the | | |
|---|---|---|---|
| resolution | present invention | PHLAT | HLAminer |
| 8-digit | 99.94% | — | — |
| 6-digit | 99.95% | 80.80% | — |
| 4-digit | 99.95% | 88.75% | 50.12% |
| 2-digit | 100% | 96.39% | 77.82% |

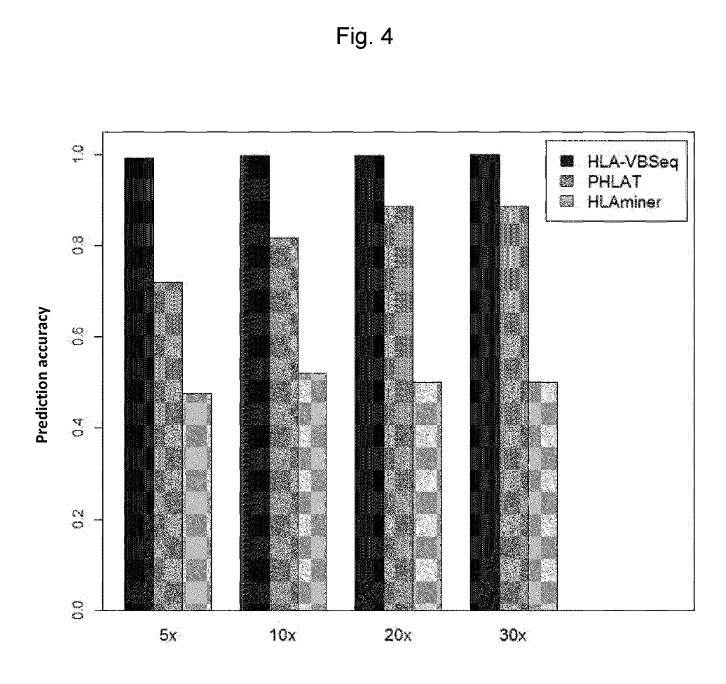

In Table 1, the system of the present invention was able to estimate the HLA type with an especially high accuracy of 99.94% at eight-digit resolution which can not be conducted with existing PHLAT and HLAminer. Comparing the estimation accuracy of the system of the present invention with that of the existing PHLAT and HLAminer, it became clear that the system of the present invention achieved better prediction performance at the four-digit resolution and the six-digit resolution than the existing software. FIG. 4 is a drawing showing prediction accuracy of HLA types at four-digit resolution at various depth of coverage in the simulation data. In FIG. 4, the prediction accuracy when using the system of the present invention was always better than the accuracy with PHLAT and HLAminer throughout the examined depth of coverage.

In particular, in the system of the present invention, HLA type prediction was made at an accuracy of 99.36% at four-digit resolution using the simulation data of average depth of 5×. In PHLAT, only SNP sites are examined in order to compute likelihood, but with only the limited information, in case if other polymorphic sites (such as deletions or insertions) are important for determining the HLA type, it is not effective. Another possible disadvantage of PHLAT is that the system needs prior information about HLA allele frequency. However, the HLA allele frequency is particularly diverse among human populations and it is not always possible to guess the racial roots of the provided gene specimens. In contrast, the system of the present invention does not require prior information on HLA allele frequency.

### (3) Real Data Analysis (1)

The system of the present invention is applied to whole-genome sequencing data of CEU trio samples (NA12878 (child), NA12891 (father) and NA12892 (mother)) used in the international HapMap project, which were not amplified by PCR method. Here, 100 bp paired-end data was generated using HiSeq2000, the average insertion length was 300 bp, and the depth of coverage of these data was 45× for each sample (all data was provided by Illumina, Inc.). Table 2 shows the predicted HLA types for the CEU trio.

TABLE 2

| Sample | Predicted HLA types | |
|---|---|---|
| NA12878 (child) | A*01:01:01:01 | A*11:01:01 |
| | B*08:01:01 | B*56:01:01 |
| | C*01:02:01 | C*07:01:01:01 |
| | DQA1*01:01:02 | DQA1*05:01:01:02 |
| | DQB1*02:01:01 | DQB1*05:01:01:02 |
| | DRB1*03:01:01:01 | DRB1*01:01:01 |
| NA12891 (father) | A*01:01:01:01 | A*24:02:01:01 |
| | B*07:02:01 | B*08:01:01 |
| | C*07:01:01:01 | C*07:02:01:03 |
| | DQA1*01:02:01:01 | DQA1*05:01:01:02 |
| | DQB1*02:01:01 | DQB1*06:02:01 |
| | DRB1*03:01:01:01 | DRB1*15:01:01:02 |
| NA12892 (mother) | A*02:01:01:01 | A*11:01:01 |
| | B*15:01:01:01 | B*56:01:01 |
| | C*01:02:01 | C*04:01:01:01 |
| | DQA1*01:01:02 | DQA1*01:01:02 |
| | DQB1*05:01:01:02 | DQB1*05:01:01:01 |
| | DRB1*01:01:01 | DRB1*01:01:01 |

In Table 2, the HLA types for the class I locus (HLA-A, -B, and -C) predicted by using the system of the present invention are matched with HLA types experimentally verified at four-digit resolution. These are shown in bold text in Table 2. Many of the HLA types were predicted at 8-digit resolution by the system of the present invention. The HLA types of HLA-A, -B and -C loci predicted in the system of the present invention are consistent with HLA types predicted with PHLAT at six-digit resolution except for “B*07:02:01” (one allele in NA12891). Another literature (Major et al., PloS one, 8(11): e78410 (2013)) also reported one of the HLA-B alleles of NA12891 as “B*07:02:01”, which is consistent with the prediction with the system of the present invention. PHLAT, on the other hand, predicted this HLA type as “B*07:02:29”. Overall, the HLA types of the trio (child, father and mother) predicted in the system of the present invention was more consistent than those predicted by PHLAT.

The HLA types of the HLA-DQA1, -DQB1 and -DRB1 loci predicted with the system of the present invention are consistent with those predicted with PHLAT at six-digit resolution, except for DQA1*01:01:02 (one allele in NA12878 and two alleles in NA12892). PHLAT predicted the HLA allele as “DQA1*01:01:01”, which was predicted as “DQA1*01:01:02” with the system of the present invention. However, its genomic sequence itself was missing in the IMGT database release 3.15.0, so it was not possible to determine the success or failure between the two systems.

### (4) Real Data Analysis (2)

The system of the present invention was applied to the 1KJPN population (1,070 healthy Japanese who participated in the cohort study of Tohoku Medical Megabank Project) and HLA allele at HLA-A, HLA-B and HLA-C loci were estimated. This example is based on mapping of sequence reads to genomic HLA allele sequences registered in the IMGT/HLA database. This analysis confirmed that it is possible to classify HLA-A alleles for 2,063 alleles out of 2,140 alleles at full resolution (eight-digits in HLA nomenclature).

Also, it is confirmed that the allele frequencies of HLA-A, HLA-B, and HLA-C at the predicted four-digit resolution (nucleotide difference that changes the amino acid sequence) are very similar to those determined at 4-digit resolution using the method PCR-SSOP (Itoh, Y. et al., Immunogenetics 57, 717-729 (2005)) in another Japanese population of 1018 people. Since both of the two Japanese populations which were compared include a sufficient number of samples of 1,000 or more, it can be assumed that the allele frequency distribution is close to the actual HLA allele frequency of a Japanese population. The fact that the estimated result with the system of the present invention and the estimated result with PCR-SSOP are very similar suggests that the allele frequency of a Japanese population could be correctly estimated at four-digit resolution whichever method was used.

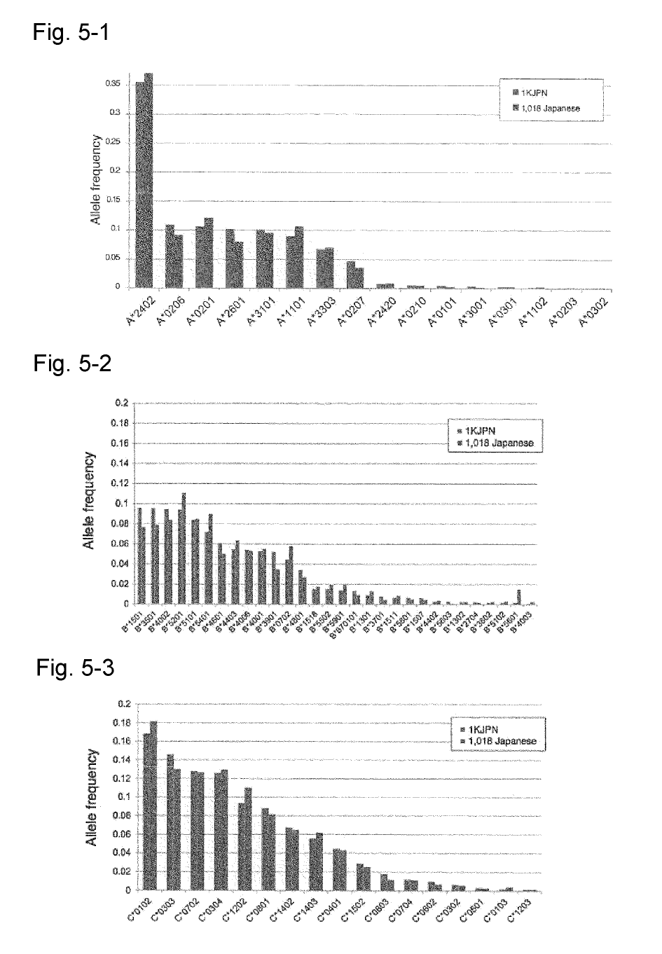

FIG. 5-1 is a graph showing the results of examining HLA allele frequencies calculated for HLA-A, FIG. 5-2 for HLA-B, and FIG. 5-3 for HLA-C with both methods. The vertical axis shows allele frequency and the horizontal axis shows HLA alleles at four-digit resolution, and a graph bar on the left side of the same HLA allele type is the analysis result estimated using the system of the present invention in this 1KJPN population, and a graph bar on the right shows the analysis result with PCR-SSOP applied to another Japanese population of 1,018 people.

### INDUSTRIAL APPLICABILITY

The system of the present invention realizes efficient and accurate genotyping of the gene loci in which there are similar nucleotide sequences in multiple locations in genome or there are many polymorphisms known, such as HLA loci, HLA being a human MHC, cytochrome P450 loci, gene loci encoding an immunoglobulin, gene loci encoding T cell receptors, gene loci encoding olfactory receptors, using whole-genome sequencing data without the need of primer design that is specific to the gene locus or prior knowledge of the allele frequency of the gene loci. Regardless of individual or population scale sequence data, it is possible to easily apply the system of the present invention for genotyping each locus at any selected gene loci, which is useful for studies to identify the relationship between genotype and phenotype and for clinical applications such as HLA type matching of donors and recipients during organ transplantation. In addition, by applying the present invention to other MHC other than HLA, for example, mammalian MHC such as H-2 (histocompatibility-2) which is a mouse MHC, and avian MHC such as chicken B locus, the present invention can be used as providing a more accurate and detailed variety discrimination, and further, providing basic knowledge for establishing therapeutic guidelines for diseases of pets and protected animals.

### EXPLANATION OF REFERENCE NUMERALS

B1-1 A box indicating the presence of read data
B1-2 A box indicating the presence of human genome reference
S1 A step of performing the first mapping
B2 A box indicating the presence of the result from the first mapping
S2-1 A step of extracting reads from the result of the first mapping
S2-2 A step of extracting unmapped reads
B3 A box indicating the presence of the read information extracted by S2
B4 A box indicating the presence of HLA allele reference sequence
S3 A step of performing the second mapping
B5 A box indicating the presence of HLA mapping read information
B6 A box indicating the presence of electronic information in which HLA type determination has been made
S4-11 A step of describing a function of retrieval for performing optimization
S4-12 A step of initializing parameters for performing optimization processes
S4-13 A step of describing a function of E step for optimization
S4-14 A step of describing a function of M step for optimization
S4-15 A step of describing a function of loop and convergence for optimization
S4-21 A step of describing a function of retrieval for performing optimization
S4-22 A step of initializing parameters for performing optimization processes
S4-23 A step of describing a function of E step for optimization
S4-24 A step of describing a function of M step for optimization
S4-25 A step of describing a function of loop and convergence for optimization
S4-31 A step of describing a function of retrieval for performing optimization
S4-32 A step of initializing parameters for performing optimization processes
S4-33 A step of describing a function of E step for optimization
S4-34 A step of describing a function of M step for optimization
S4-35 A step of describing a function of loop and convergence for optimization
S5-1 A step of performing calculation of individual depth of coverage
S5-2 A step of performing selection of the two with the largest individual depth of coverage
S5-3 A step of performing selection based on the rejection depth and the largest individual depth of coverage
D5-1 A box indicating a conclusion that the HLA type is not determined
S5-4 A step of performing selection based on the rejection depth and the second largest individual depth of coverage
S5-5 A step performed when the second largest individual depth of coverage is smaller than the rejection depth
D5-2 A box indicating a conclusion that it is homozygous of the HLA allele with the largest individual depth of coverage
D5-3 A box indicating a conclusion that it is heterozygous of the HLA alleles with the largest individual depth of coverage and another allele that is not determined
S5-6 A step performed when the second largest individual depth of coverage is not smaller than the rejection depth
D5-4 A box indicating a conclusion that it is homozygous of the HLA allele with the largest individual depth of coverage
D5-5 A box indicating a conclusion that it is heterozygous of the HLA alleles with the largest and the second largest individual depth of coverage

1. A method for optimizing read information of DNA, which is characterized by executing all or a part of the following steps (1) to (6) using read information which contains mapping correspondence of each read to alleles of selected gene loci or an individual gene locus obtained by mapping a nucleotide sequence of read data where read information of DNA derived from alleles of the gene loci or an individual gene locus is mixed:
(1) a step in which, for each read, quantification of an expected number of mappings to each allele of the gene loci or individual gene locus, is performed;
(2) a step in which the expected number of mappings quantified in step (1) is summed for each allele of the gene loci or individual gene locus to calculate a total number of expected mappings;
(3) a step in which the total number of expected mappings calculated in step (2) is divided by the sum of the total number of expected mappings of all the alleles of the gene loci or individual gene locus, to calculate a fraction of reads allocated to each allele of the gene loci or individual gene locus relative to a total read amount mapped to alleles of the gene loci or individual gene locus;
(4) a step in which the fraction of reads obtained in step (3) is assigned to each allele of the gene loci or individual gene locus as frequency, and based on the assigned frequencies, for each read, the expected number of mappings to each allele of the gene loci or individual gene locus is calculated again;
(5) a step in which step (2) or (3) is executed again based on the updated expected number of mappings obtained in step (4), to calculate a fraction of the number of reads allocated to an allele of the gene loci or individual gene locus relative to the total read amount mapped to alleles of the gene loci or individual gene locus; and
(6) a step in which steps (4) and (5) are repeatedly executed, until difference between the expected number of mappings to each allele of the gene loci or individual gene locus calculated in step (4) and that calculated in the previous step (4) is not recognized for all the reads, or difference between the fraction of reads calculated in step (5) and that calculated in the previous step (5), is not recognized for all the alleles in the gene loci or individual gene locus, and the converged expected number of mappings for each read for each allele of the gene loci or individual gene locus, or, the converged fraction of reads for alleles of the gene loci or individual gene locus, is recognized as optimized data.

2. An optimization method in which mapping of the read information of DNA from a subject to alleles of selected gene loci or an individual gene locus is optimized by a computer, which includes a step to determine for each read an expected number of mappings to alleles of the gene loci or individual gene locus, and a step to estimate allele frequencies θ of the gene loci or individual gene locus, θ being an objective parameter, where θ is T dimensional vector, the T being the number of alleles of the gene loci or individual gene locus, given nucleotide sequences of all reads in data in which read information of DNA derived from alleles of the gene loci or individual gene locus is mixed as observed data R, and which is characterized in that, with respect to (a) a latent variable Tₙ dependent on θ with respect to allele selection of the gene loci or individual gene locus of read n, and (b) a latent variable Sₙ dependent on Tₙ with respect to starting position of read n, and given nucleotide sequence of read n as observed data Rₙ, at least (i) variables Tₙ and Sₙ or (ii) variable Tₙ is incorporated to calculate an estimate of the parameter in an estimation process of the objective parameter θ, so that observed data Rₙ depends on the latent variables during the estimation process of the objective parameter θ from observed data Rₙ.

3. The optimization method according to claim 1 or 2, which is characterized by calculating for each read the expected number of mappings to each allele of the selected gene loci or individual gene locus, and calculating estimated value of allele frequency 0 of the gene loci or individual gene locus, θ being an objective parameter, by executing steps based on a maximum likelihood estimation method, or Bayesian estimation method.

4. The optimization method according to claim 2 or 3, which is characterized by using an indicator variable Zₙₜₛ (Zₙₜₛ equals to one if (Tₙ, Sₙ)=(t, s), and zero otherwise), which summarizes latent variables Tₙ and Sₙ, or Zₙₜ (Zₙₜ equals to one if Tₙ=t, and zero otherwise), which summarizes the latent variable Tₙ.

5. The optimization method according to claim 4, which is characterized by executing the following steps (1) to (5):
(1) a step in which a first update value of a posterior probability of Zₙₜₛ=1 or Zₙₜ=1 is calculated based on a given initial value of θₜ, and a first updated value of a maximum likelihood estimate of θₜ is further calculated, based on the first updated value of the posterior probability of Zₙₜₛ=1 or Zₙₜ=1, or, (2) a step in which a first updated value of a maximum likelihood estimate of θₜ is calculated, based on a given initial value of a posterior probability of Zₙₜₛ=1 or Zₙₜ=1;
(3) a step in which an updated value of the posterior probability of Zₙₜₛ=1 or Zₙₜ=1 is calculated, based on the updated value of the maximum likelihood estimate of θₜ calculated by a previous step (4) (however, for the first time, by step (1) or step (2));
(4) a step in which an updated value of the maximum likelihood estimate of θₜ is calculated, based on the updated value of the posterior probability of Zₙₜₛ=1 or Zₙₜ=1 calculated in step (3); and
(5) (i) a step in which log likelihood is calculated based on the updated value of the posterior probability of Zₙₜₛ=1 or Zₙₜ=1 calculated in step (3) and the updated value of the maximum likelihood estimate of θₜ calculated in step (4), to evaluate convergence of the log likelihood,
(ii) a step in which convergence of the updated value of the posterior probability of Zₙₜₛ=1 or Zₙₜ=1 calculated in step (3) is evaluated, or
(iii) a step in which convergence of the updated value of the maximum likelihood estimate of θₜ calculated in step (4), and,
if convergence is recognized, θₜ in each step is determined as a final estimate value, and if convergence is not recognized, iteration of steps (3), (4) and (5) is determined.

6. The optimization method according to claim 4, which is characterized by executing the following steps (1) to (5):
(1) a step in which a first update posterior distribution of Zₙₜₛ or Zₙₜ is calculated based on a given initial posterior distribution of θₜ based on the hyperparameter α₀ representing prior information of allele frequencies of the selected gene loci or individual gene locus, and a first updated posterior distribution of θₜ is further calculated based on the first updated posterior distribution of Zₙₜₛ or Zₙₜ, or,
(2) a step in which an updated posterior distribution of θₜ is calculated based on a given initial distribution of the Zₙₜₛ or Zₙₜ;
(3) a step in which an updated posterior distribution of Zₙₜₛ or Zₙₜ is calculated, based on the updated posterior distribution of θₜ, calculated by the previous step (4) (however, for the first time, by step (1) or step (2));
(4) a step in which an updated posterior distribution of θₜ is calculated, based on the updated posterior distribution of Zₙₜₛ or Zₙₜ calculated in step (3); and
(5) a step in which convergence of the updated posterior distribution of θₜ calculated in step (4) is evaluated, and if convergence is recognized, the expected value of θₜ is determined as a final estimate value, and if convergence is not recognized, iteration of steps (3), (4) and (5) is determined.

7. The optimization method according to any one of claims 1 to 6, in which the data in which read information of DNA derived from alleles of the selected gene loci or individual gene locus is mixed, is read information in which mapping of each read to each allele of the gene loci or individual gene locus is identified by mapping read information of a subject to reference nucleotide sequences of alleles of the gene loci or individual gene locus registered in a database, and the mapping is characterized by executing the following steps (a) and (b):
(a) a step in which with respect to nucleotide sequence information of reads obtained from the subject, reads are mapped to a human genome nucleotide sequence, and then those mapped to alleles of the gene loci or individual gene locus are extracted; and
(b) a step in which the read sequence information mapped to alleles of the gene loci or individual gene locus obtained in step (a) is mapped to the reference nucleotide sequences of alleles of the gene loci or individual gene locus registered in a database, and reads mapped to each allele of the gene loci or individual locus are extracted, and read information is obtained, in which mapping of each read to each allele of the gene loci or individual gene locus is identified.

8. The optimization method according to claim 7, which is characterized in that the mapping performed in steps (a) and (b) allows one read to be mapped to multiple alleles of the selected gene loci or individual gene locus.

9. The optimization method according to claim 7 or 8, which is characterized in that in addition to the reads mapped to alleles of the selected gene loci or individual gene locus obtained in step (a), reads that are not mapped to whole human genome are extracted, and it becomes the target of the re-mapping in step (b).

10. The optimization method according to any one of claims 1 to 9, which is characterized in that the selected gene loci or individual gene locus are MHC gene loci or individual MHC gene locus.

11. The optimization method according to claim 10, which is characterized in that MHC is HLA.

12. A genotype determination method for selected gene loci or an individual gene locus, which is characterized by calculating individual depth of coverage of reads for each allele of the gene loci or individual gene locus from allele frequency of the selected gene loci or individual gene locus obtained from the optimization method recited in any one of claims 1 to 11, selecting top two or less than two alleles of the gene loci or individual gene locus from a list of alleles that are sorted based on the individual depth of coverage by descending order, and determining the alleles as candidates for genotyping of each gene locus of the selected gene loci or the individual gene locus.

13. A genotype determination method for selected gene loci or an individual gene locus, in which individual depth of coverage of reads for each allele of the gene loci or individual gene locus is calculated from allele frequency of the gene loci or individual gene locus obtained from the optimization method recited in any one of claims 1 to 12, top two or less than two alleles of the gene loci or individual gene locus are selected from a list of alleles that are sorted based on the individual depth of coverage by descending order, and the alleles are determined as candidates for genotyping of each gene locus of the selected gene loci or the individual gene locus, and which is characterized by setting a threshold of depth of coverage of alleles at 5-50% of the depth of coverage of total reads as a rejection threshold, and eliminating alleles of the gene loci or gene locus whose individual depth of coverage are less than the threshold from candidates for genotyping of each gene locus of the selected gene loci or the individual gene locus.

14. The determination method according to claim 13, which is characterized in that after eliminating the alleles from candidates for genotyping of each gene locus of the selected gene loci or the individual gene locus, the following (i) or (ii) is determined:
(i) for a case that there is one allele that is selected for genotyping of the gene locus of the gene loci or the individual gene locus, if the individual depth of coverage of the allele is twice or more of the rejection threshold, it is determined that the genotype is homozygous of the allele, and if it is less than twice of the rejection threshold, it is determined that the genotype is heterozygous of the allele, and
(ii) for the case that there are two alleles that are selected for genotyping of the gene locus of the gene loci or the individual gene locus, if the individual depth of coverage of the one whose individual depth of coverage is larger is less than twice of the smaller one, it is determined that the genotype is heterozygous of the two alleles, or if the individual depth of coverage of the one whose individual depth of coverage is larger is twice or more of the smaller one, it is determined that the genotype is homozygous of the allele whose individual depth of coverage is larger.

15. The genotype determination method according to any one of claims 12 to 14, which is characterized by that the selected gene loci or individual gene locus is MHC gene loci or individual MHC gene locus.

16. The genotype determination method according to claim 15, which is characterized in that MHC is HLA.

17. A computer system for optimizing read information in which read mapping information of each read to each allele of gene loci or an individual locus to be optimized is identified by mapping nucleotide sequence of reads in data in which read information of DNA derived from alleles of the gene loci or individual gene locus is mixed, comprising a recording unit and an arithmetic processing unit, which is characterized in that all or a part of the following processes (A) to (G) is executed:
(A) in the recording unit, read information of DNA derived from a subject is recorded as data of nucleotide sequence of read and alleles of the gene loci or individual gene locus to which the read is mapped;
(B) in the arithmetic processing unit, based on the information of the recording section, for each read, an expected number of mappings for each allele of the gene loci or individual gene locus is calculated by executing numerical processing;
(C) for each allele, the expected number of mappings calculated in the process (B) is summed for each allele of the gene loci or individual gene locus to calculate a total number of expected mappings;
(D) for each allele, the total number of expected mappings calculated in the process (C) is divided by the sum of the total number of expected mappings for all alleles of the gene loci or individual gene loci, and the process of calculating the fraction of reads allocated to each allele of the gene loci or individual gene locus with respect to the total amount of reads mapped to alleles of the gene loci or individual gene locus is executed;
(E) the fraction of reads calculated in the above process (C) is assigned as frequency to each allele of the gene loci or individual gene locus, and given the assignment frequency, the process (B) is executed again in which for each read, the expected number of mappings to each allele of the gene loci or individual gene locus is calculated;
(F) the process (C) or (D) is executed again for the new expected number of mappings calculated by the process (E), and the process of calculating the fraction of reads allocated to each allele of the gene loci or individual gene locus with respect to the total amount of reads mapped to alleles of the gene loci or the individual locus is executed; and
(G) the processes (E) and (F) are repeatedly executed, until for all reads, difference between the number of expected mappings to each allele of the gene loci or individual gene locus calculated in the process (E) and that calculated in the previous process (E) is not recognized, or for all alleles of the gene loci or individual gene locus, difference between the fraction of reads calculated in the process (F) and that calculated in the previous process (F) is not recognized, and a converged number of expected mappings for each read to each allele of the gene loci or individual gene locus, or the converged value of the fraction of reads for each allele of the gene loci or individual gene locus, is recognized as the optimized data.

18. A computer system for optimizing, for each read, the number of expected mappings to each allele of selected gene loci or individual gene locus for data in which read information of DNA derived from alleles of the gene loci or individual locus is mixed, comprising a recording unit and an arithmetic processing unit, which is characterized in that all or a part of the following processes (A) to (E) is executed:
(A) in the recording unit, read information of DNA derived from a subject is recorded as observed data of nucleotide sequence of read and alleles of the gene loci or individual gene locus to which the read is mapped;
(B) in the arithmetic processing unit, based on the information of the recording unit, either one of the following initialization processes (B)-1 and (B)-2 is executed:
(B)-1: the process of calculating an initial value of θ, which represents allele frequency of the gene loci or individual gene locus, and
(B)-2: the process of calculating initial values of two latent variables, (a) and (b) described below, that are related to the θ described above and the data of DNA read information of the subject, which is the observed data:
(a) a variable Tₙ that is dependent on θ, which represents allele selection of read n in the gene loci or individual gene locus,
(b) a posterior probability of an indicator variable Zₙₜₛ (Zₙₜₛ equals to one if (Tₙ, Sₙ)=(t, s), and zero otherwise), which summarizes Sₙ and a start position of read n that is dependent on Tₙ, or Zₙₜ (Zₙₜ equals to one if Tₙ=t, and zero otherwise), which summarizes the latent variable Tₙ;
(C) in the arithmetic processing unit, based on the parameter θ calculated in the process (B)-1, the calculation process of the posterior probability of indicator variable Zₙₜₛ or Zₙₜ=1 is executed;
(D) in the arithmetic processing unit, a first updated value of the maximum likelihood estimate of parameter θ is calculated based on the posterior probability of indicator variable Zₙₜₛ or Zₙₜ=1 calculated in the process (B)-2 or (C); and
(E) in the arithmetic processing unit, the processes (C) and (D) are executed again based on the first updated value of the maximum likelihood estimate of parameter θ calculated in the process (D), and an iterative loop process for calculating a second updated value of parameter θ is repeatedly executed, until difference between the new updated value of θ and the previous updated value of θ is not recognized, and the converged parameter θ is recorded as an optimized value in the recording unit.

19. A computer system for optimizing, for each read, the number of expected mappings to each allele of selected gene loci or an individual gene locus for data where read mapping information of the gene loci or individual gene locus is mixed, comprising a recording unit and an arithmetic processing unit, which is characterized in that all or a part of the following processes (A) to (E) is executed:
(A) in the recording unit, read information of DNA derived from the subject is recorded as observed data of nucleotide sequence of read and alleles of the gene loci or individual gene locus to which the read is mapped,
(B) in the arithmetic processing unit, based on the observed data retrieved from the recording unit, either one of the following initialization processes (B)-1 and (B)-2 is executed:
(B)-1: the process of calculating an initial value of posterior distribution of θₜ, based on an initial value of hyperparameter αₜ which represents prior information of allele frequency of the gene loci or individual gene locus, and
(B)-2: the process of calculating initial distribution of two latent variables, (a) and (b) described below, that are related to the distribution of θ described-above and the data of DNA read information of the subject, which is observed data:
(a) a variable Tₙ that is dependent on θ, which represents allele selection of read n in the gene loci or individual gene locus, and
(b) posterior distribution of an indicator variable Zₙₜₛ (Zₙₜₛ equals to one if (Tₙ, Sₙ)=(t, s), and zero otherwise), which summarizes Sₙ, the start position of read n that is dependent on Tₙ, or Zₙₜ (Zₙₜ equals to one if Tₙ=t, and zero otherwise), which summarizes the latent variable Tₙ;
(C) in the arithmetic processing unit, based on the distribution of parameter θ calculated in the process (B)-1, the calculation process of the posterior distribution of indicator variable Zₙₜₛ or Zₙₜ is executed;
(D) in the arithmetic processing unit, the first updated posterior distribution of parameter θ is calculated based on the posterior distribution of Zₙₜₛ or Zₙₜ calculated in the process (B)-2 or (C),
(E) in the arithmetic processing unit, the processes (C) and (D) are executed again based on the first updated value of the posterior distribution of θ, and an iterative loop process for calculating a second updated posterior distribution of θ is repeatedly executed, until difference between the expected value of the new updated posterior distribution of θ and the expected value of the previous updated posterior distribution of θ is not recognized, and the converged expected value of the posterior distribution of θ is recorded as the optimized value in the recording unit.

20. The computer system according to any one of claims 17 to 19, which is characterized in that the data in which read information of DNA derived from alleles of the selected gene loci or individual gene locus is mixed is read information in which mapping of each read to each allele of the gene loci or individual gene locus is identified by mapping read information of the subject to nucleotide sequences of alleles of the gene loci or individual gene locus registered in a database, and the mapping is executed by the following processes (a) and (b):
(a) a process in which with respect to nucleotide sequence information of reads obtained from a subject, reads are mapped to human genome nucleotide sequence, and then those mapped to alleles of the gene loci or individual gene locus are extracted; and
(b) a process in which the read sequence information mapped to alleles of the gene loci or individual gene locus obtained in process (a) is mapped to reference nucleotide sequences of alleles of the gene loci or individual gene locus that are registered in a database, and read information is obtained, in which mapping information and mapping states for each read to each allele of the gene loci or individual gene locus are identified.

21. The computer system according to claim 20, which is characterized in that the mapping performed in process (b) allows one read to be mapped to multiple alleles of the selected gene loci or individual gene locus.

22. The computer system according to claim 20 or 21, which is characterized in that in addition to the reads mapped to alleles of the selected gene loci or individual gene locus obtained in process (a), reads that are not mapped to the whole human genome are extracted, and it becomes the target of the re-mapping in process (b).

23. A computer system for determining genotype of each gene locus of selected gene loci or individual gene locus of a subject, comprising a recording unit and an arithmetic processing unit, which is characterized in that all or a part of the following processes (α) to (δ) is executed:
(α) in the recording unit, at least allele frequencies and depth of coverage of total reads of the gene loci or individual gene locus of the subject that are obtained by the optimization method recited in any one of claims 1 to 11 are recorded;
(β) in the arithmetic processing unit, based on the allele frequencies of the gene loci or the individual gene locus recorded in the recording unit, processing of calculating individual depth of coverage of each allele of the gene loci or individual gene locus, and processing of allocating the calculated individual depth of coverage to the alleles of the gene loci or the individual gene locus, are executed;
(γ) given a rejection threshold value, which is set as a frequency number of 5 to 50% of the average depth of coverage of all reads, a process in which alleles of the gene loci or individual gene locus whose individual depth of coverage is smaller than the threshold value are excluded from candidates for genotyping, is executed;
(δ):
(δ)-1 after the exclusion process of (γ), for a case that there is one allele that is selected for genotyping the gene locus of the selected gene loci or the individual gene locus, if the individual depth of coverage of the allele is twice or more of the rejection threshold value, it is determined that the genotype is homozygous of the allele, and if it is less than twice of the rejection threshold value, it is determined that the genotype is heterozygous of the allele, and
(δ)-2 after the exclusion process of (γ), for a case that there are two alleles that are selected for genotyping the gene locus of the selected gene loci or the individual gene locus, if the individual depth of coverage of the one whose individual depth of coverage is larger is less than twice of the smaller one, it is determined that the genotype is heterozygous of the two alleles, or if the individual depth of coverage of the one whose individual depth of coverage is larger is twice or more of the smaller one, it is determined that the genotype is homozygous of the allele whose individual depth of coverage is larger.

24. The computer system according to any one of claims 17 to 23, which is characterized in that the selected gene loci or individual gene locus is MHC gene loci or individual MHC gene locus.

25. The computer system according to claim 24, which is characterized in that MHC is HLA.

26. A computer program for optimizing read information in which mapping correspondence of each read to alleles of a selected gene loci or an individual gene locus is obtained by mapping a nucleotide sequence of read data where read information of DNA derived from alleles of the selected gene loci or an individual gene locus is mixed, which is characterized by including algorithms to realize all or a part of the following functions (1) to (7) in a computer:
(A) the function (1) in which read information of DNA derived from a subject recorded in a recording unit as data of nucleotide sequence of read and alleles of the gene loci or individual gene locus to which the read is mapped is retrieved from the recording unit;
(B) the function (2) in which based on the read information retrieved in the function (1), for each read, an expected number of mappings for each allele of the gene loci or individual gene locus is calculated by executing numerical processing;
(C) the function (3) in which for each allele, the expected number of mappings quantified in the function (2) is summed for each allele of the gene loci or individual gene locus to calculate the total number of expected mappings;
(D) the function (4) in which for each allele, the total number of expected mappings calculated in the function (3) is divided by the sum of the total number of expected mappings for all alleles of the gene loci or individual gene loci, and the process of calculating the fraction of reads allocated to each allele of the gene loci or individual gene locus with respect to the total amount of reads mapped to alleles of the gene loci or individual gene locus is executed;
(E) the function (5) in which the fraction of reads calculated in the function (4) is assigned as frequency to each allele of the gene loci or individual gene locus, and given the assignment frequency, the function (2) is executed again in which for each read, the expected number of mappings to each allele of the gene loci or individual gene locus is calculated;
(F) the function (6) in which the function (3) or (4) is executed again for the newly obtained expected number of mappings calculated by the function (5), and the process of calculating the fraction of reads allocated to each allele of the gene loci or individual gene locus with respect to the total amount of reads mapped to alleles of the gene loci or the individual locus is executed; and
(G) the function (7) in which the functions (5) and (6) are repeatedly executed, until for all reads, difference between the number of expected mappings to each allele of the gene loci or individual gene locus calculated in the function (5) and that calculated in the previous function (5), is not recognized, or for all alleles of the gene loci or individual gene locus, difference between the value of the fraction of reads calculated in the function (6) and that calculated in the previous function (6), is not recognized, and the converged number of expected mappings for each read to each allele of the gene loci or individual gene locus, or the converged value of the fraction of reads for each allele of the gene loci or individual gene locus, is recognized as an optimized data.

27. A computer program for optimizing, for each read, the number of expected mappings to each allele of selected gene loci or individual gene locus for data in which read information of DNA derived from alleles of the gene loci or gene locus is mixed, which is characterized by including algorithms for realizing all or a part of the following functions 1 to 5:
(A) function 1 in which read information of DNA derived from a subject recorded in a recording unit as observed data of nucleotide sequence of read and alleles of the gene loci or individual gene locus to which the read is mapped, is retrieved from the recording unit;
(B) function 2 in which based on the observed data retrieved by function 1, either one of the following initialization processes (B)-1 and (B)-2 is executed:
(B)-1: the process of calculating an initial value of θ, which represents allele frequency of the gene loci or individual gene locus, and
(B)-2: the process of calculating initial values of two latent variables, (a) and (b) described below, that are related to the θ described above and the data of DNA read information of the subject, which is the observed data:
(a) a variable Tₙ that is dependent on θ, which represents allele selection of read n in the gene loci or individual gene locus, and
(b) posterior probability of an indicator variable Zₙₜₛ (Zₙₜₛ equals to one if (Tₙ, Sₙ)=(t, s), and zero otherwise), which summarizes Sₙ which is start position of read n that is dependent on Tₙ, or Zₙₜ (Zₙₜ equals to one if Tₙ=t, and zero otherwise), which summarizes a latent variable Tₙ;
(C) function 3 in which based on the parameter θ calculated in the process (B)-1, the calculation process of the posterior probability of indicator variable Zₙₜₛ or Zₙₜ=1 is executed;
(D) function 4 in which a first updated value of maximum likelihood estimate of parameter θ is calculated based on the posterior probability of Zₙₜₛ or Zₙₜ=1 calculated in process (B)-2 or function 3; and
(E) function 5 in which function 3 and 4 are executed again based on the first updated value of the maximum likelihood estimate of θ, and the iterative loop process for calculating a second updated value of θ, is repeatedly executed, until difference between the new updated value of θ and the previous updated value of θ is not recognized, and the converged parameter θ is recorded as an optimized value in the recording unit.

28. A computer program for optimizing, for each read, the number of expected mappings to each allele of selected gene loci or individual gene locus for data in which read information of DNA derived from alleles of the gene loci or gene locus is mixed, which is characterized by including algorithms for realizing all or a part of the following functions 1 to 5 in a computer:
(A) function 1 in which read information of DNA derived from a subject recorded in a recording unit as observed data of nucleotide sequence of read and alleles of the gene loci or individual gene locus to which the read is mapped, is retrieved from the recording unit;
(B) function 2 in which based on the observed data retrieved by function 1, either one of the following initialization processes (B)-1 and (B)-2 is executed:
(B)-1: a process of calculating an updated value of posterior distribution of θₜ, based on an initial value of hyperparameter αₜ which represents prior information of allele frequency of the gene loci or individual gene locus, and
(B)-2: a process of calculating initial distributions of two latent variables, (a) and (b) described below, that are related to the distribution of θ described above and the data of DNA read information of the subject, which is observed data:
(a) a variable Tₙ that is dependent on θ, which represents allele selection of read n in the gene loci or individual gene locus,
(b) posterior distribution of indicator variable Zₙₜₛ (Zₙₜₛ equals to one if (Tₙ, Sₙ)=(t, s), and zero otherwise), which summarizes Sₙ which is start position of read n that is dependent on Tₙ, or Zₙₜ (Zₙₜ equals to one if Tₙ=t, and zero otherwise), which summarizes latent variable Tₙ;
(C) function 3 in which based on the distribution θ calculated in the process (B)-1, the calculation process of the posterior distribution of indicator variable Zₙₜₛ or Zₙₜ is executed;
(D) function 4 in which a first updated value of the posterior distribution of θ is calculated based on the posterior distribution of Zₙₜₛ or Zₙₜ calculated in function (B)-2 or function 3; and
(E) function 5 in which function 3 and 4 are executed again based on the first updated value of the posterior distribution of θ, and an iterative loop process for calculating a second updated value of the posterior distribution of θ is repeatedly executed, until difference between an expected value of the new updated posterior distribution of θ and an expected value of the previous updated posterior distribution of θ is not recognized, and the converged expected value of posterior distribution of θ is recorded as an optimized value in the recording unit.

29. The computer program according to any one of claims 26 to 28, in which the data in which read information of DNA derived from alleles of the selected gene loci or individual gene locus is mixed, is read information in which mapping of each read to each allele of the gene loci or individual gene locus is identified by mapping read information of the subject to nucleotide sequences of alleles of the gene loci or individual gene locus registered in a database, which is characterized in that the mapping is realizing by executing the following functions (a) and (b):
(a) a function in which with respect to nucleotide sequence information of reads obtained from the subject, reads are mapped to human genome nucleotide sequence, and then those mapped to alleles of the gene loci or individual gene locus are extracted; and
(b) a function in which with respect to the read sequence information mapped to alleles of the gene loci or individual gene locus obtained in function (a), reads are mapped to reference nucleotide sequences of alleles of the gene loci or individual gene locus that are registered in a database, and reads mapped to each allele of the gene loci or individual locus are extracted, and read information is obtained, in which mapping of each read to each allele of the gene loci or individual gene locus is identified.

30. The computer program according to claim 29, which is characterized in that the mapping performed in function (b) allows one read to be mapped to multiple alleles of the selected gene loci or individual gene locus.

31. The computer program according to claim 29 or 30, which is characterized in that in addition to the reads mapped to alleles of the selected gene loci or individual gene locus obtained in function (a), reads that are not mapped to the whole human genome are extracted, and it becomes the target of the re-mapping in function (b).

32. A computer program for determining genotype of a gene locus of selected gene loci or an individual gene locus of a subject, which is characterized by including algorithms for realizing the following functions (α) to (δ) in a computer:
(α) a function α, in which at least allele frequencies and depth of coverage of total reads of the gene loci or individual gene locus of the subject that are obtained by executing the computer program recited in any one of claims 26 to 31, are retrieved;
(β) a function β, in which based on the allele frequencies of the gene loci or the individual gene locus retrieved by executing function α, calculation of individual depth of coverage of each allele of the gene loci or individual gene locus, and allocation of the calculated individual depth of coverage to the alleles of the gene loci or the individual gene locus, are executed;
(γ) a function γ, in which given a rejection threshold value, which is set as a frequency number of 5 to 50% of average depth of coverage of all reads, a process in which alleles of the gene loci or individual gene locus whose individual depth of coverage specified by the function β is smaller than the threshold value are excluded from candidates for genotyping, is executed; and
(δ): a function δ specified as (δ)-1 and (δ)-2 below:
(δ)-1 after the exclusion process in function (γ), for a case that there is one allele that is selected for genotyping the gene locus of the selected gene loci or the individual gene locus, if the individual depth of coverage of the allele is twice or more of the rejection threshold value, a process in which it is determined that the genotype is homozygous of the allele, and if it is less than twice of the rejection threshold value, it is determined that the genotype is heterozygous of the allele, and
(δ)-2 after the exclusion process in function (γ), for a case that there are two alleles that are selected for genotyping the gene locus of the selected gene loci or the individual gene locus, if the individual depth of coverage of the one whose individual depth of coverage is larger is less than twice of the smaller one, a process in which it is determined that the genotype is heterozygous of the two alleles, or if the individual depth of coverage of the one whose individual depth of coverage is larger is twice or more of the smaller one, it is determined that the genotype is homozygous of the allele whose individual depth of coverage is larger, is executed.

33. The computer program according to any one of claims 26 to 32, which is characterized in that the selected gene loci or individual gene locus is MHC gene loci or individual MHC gene locus.

34. The computer program according to claim 33, which is characterized in that MHC is HLA.

35. A computer-readable recording medium, wherein the computer program recited in any one of claims 26 to 34 is recorded.
